SEQUENCE LISTING (2 sequences)

SEQ ID NO: 1 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 2 - protein, 140 aa, Rattus norvegicus
MDVFMKGLSKAKEGVVAAAEKTKQGVAEAAGKTKEGVLYVGSKTKEGVVHGVTTVAEKTKEQVTNVGGAVVTGVTAVAQKTVEGAGNIAAATGFVKKDQMGKGEEGYPQEGILEDMPVDPSSEAYEMPSEEGYQDYEPEA

(19) United States
(12) Patent Application Publication
(10) Pub. No.: US 2019/0194324 A1
(43) Pub. Date: Jun. 27, 2019

(54) THERAPEUTIC USES OF LAG3 THE (alpha)-SYNUCLEIN TRANSMISSION RECEPTOR

(71) Applicants: THE JOHNS HOPKINS UNIVERSITY, Baltimore, MD (US); University of Pittsburgh - Of the Commonwealth System of Higher Education, Pittsburgh, PA (US)

(72) Inventors: Ted M. Dawson, Baltimore, MD (US); Valina L. Dawson, Baltimore, MD (US); Han Seok Ko, Lutherville, MD (US); Xiaobo Mao, Timonium, MD (US); Dario Angelo Alberto Vignali, Pittsburgh, PA (US); Creg J. Workman, Franklin Park, PA (US)

(21) Appl. No.: 16/327,046

(22) PCT Filed: Aug. 22, 2017
(86) PCT No.: PCT/US2017/047878

§ 371 Date: Feb. 21, 2019

Related U.S. Application Data
(60) Provisional application No. 62/378,436, filed on Aug. 23, 2016.
(60) Provisional application No. 62/401,315, filed on Sep. 29, 2016.

(51) Int. Cl. C07K 16/28; A61P 25/28; A61P 21/00; C12N 15/113

(52) U.S. Cl. CPC C07K 16/2803; A61P 25/28; C07K 2317/76; C12N 15/1138; C12N 2310/11; A61P 21/00

(57) ABSTRACT

Described are methods of inhibiting neurodegeneration in a subject by administering to the subject an agent that prevents (alpha)-syn PFF from binding to its receptor. The agent may be a small molecule chemical compound, antibody, nucleic acid molecule, or polypeptide. Drug screening methods are also provided.

### REFERENCE TO RELATED APPLICATIONS

This application claims the benefit of U.S. Provisional Patent Application Nos. 62/378,436, filed on Aug. 23, 2016, and 62/401,315 filed on Sep. 29, 2016 both of which are hereby incorporated by reference for all purposes as if fully set forth herein.

### STATEMENT OF GOVERNMENTAL INTEREST

This invention was made with government support under grant no. NS038377 awarded by the National Institutes of Health. The government has certain rights in the invention.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Jul. 26, 2017, is named P13931-03_SL.txt and is 6,197 bytes in size.

### BACKGROUND OF THE INVENTION

Parkinson's disease (PD) is the second most common neurodegenerative disorder that is characterized clinically by motor dysfunction and pathologically by the aggregation and accumulation of α-synuclein (α-syn). Emerging evidence suggests that α-syn spreads from neuron to neuron via self-amplification, propagation, and transmission in the pathogenesis of PD. In the brains of PD patients, α-syn aggregates seem to spread in a stereotypical and topographical pattern. Postmortem examination of fetal grafts in patients with PD found α-syn positive Lewy bodies suggestive of spread of α-syn from host to graft. Other proteins such as β-amyloid and tau in Alzheimer's disease are also thought to propagate and spread and contribute to the onset and progression of this disorder. Pathological α-syn has been shown to spread among neighboring cells and/or anatomically connected brain regions. Recently recombinant α-syn pre-formed fibrils (PFF) provide a model system enabling the study of the transmission of misfolded α-syn from neuron to neuron both in vitro and in vivo. How pathological α-syn exits cells and enters neighboring neurons is not known, but entry into neurons is thought to occur through an active endocytic process. Understanding this process would enable the development of drugs for the treatment or prevention of Parkinson's disease.

### SUMMARY OF THE INVENTION

One embodiment of the present invention is a method of inhibiting neurodegeneration in a subject comprising administering to the subject an agent that prevents α-syn PFF from binding to its receptor. The methods of the present invention may be used to treat or prevent Parkinson's disease, Diffuse Lewy Body Disease (DLB), dementia with Lewy Bodies, multiple atrophy, or other neurodegenerative disease. The agent may be a small molecule chemical compound, antibody, nucleic acid molecule, or polypeptide. A suitable agent may be a vector that expresses antisense LAG3 mRNA in the subject. An agent may also be a capture molecule such as an aptamer, monoclonal antibody, antibody, or portion thereof that binds to a target molecule such as α-syn PFF. A suitable α-syn PFF receptor is lymphocyte-activation gene 3 (LAG3), Neurexin1β, Neurexin2β, Neurexin3β, or a combination thereof. Alternatively a receptor maybe an amyloid precursor-like protein 1 (APLP1). The receptor is found within a subject. A suitable subject of the present invention is a human. Alternatively, the agent may bind to α-syn PFF receptor such as LAG3. For example, a capture molecule may bind to LAG3 and prevent the binding of α-syn PFF with LAG3. An agent, such as a capture molecule, may bind to the LAG3 D1 domain specifically to amino acids 81-109, amino acids 52-80, or to both sites. An agent may also inhibit the phosphorylation of α-syn at serine 129 in a subject. Subjects suitable for the present invention comprises α-syn PFF and endocytosis of α-syn PFF is inhibited in the subject when the methods of the present invention are performed. Agents of the present invention may also inhibit the misfolding of α-syn protein in a subject. The methods of the present invention may treat and prevent Parkinson's disease, or neurological disease, in subjects.

Another embodiment of the present invention is a method of drug screening comprising the steps of: providing one or more agent(s); applying the one or more agents to LAG3; and identifying those agents that prevent α-syn PFF from binding to LAG3.

Another embodiment of the present invention is a method of drug screening comprising the steps of: providing one or more agent(s); applying the one or more agents to cells, and identifying those agents that prevent α-syn PFF from binding to LAG3 or that inhibit the phosphorylation of α-syn PFF at serine 129.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “APLP1” is meant amyloid beta precursor like protein 1. An APLP1 protein is expressed from an APLP1 gene such as a human APLP1 gene including NCB1 Gene ID: 333, as an example. An example of an APLP1 human protein sequence includes NCBI reference sequences NP-001019978.1 and NP_005157.1.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “LAG3 gene” is meant lymphocyte activation gene 3 gene and an example of such a gene is a Homo sapiens LAG 3 gene sequence having an NCBI Gene ID 3902 and a NCBI Reference Sequence number NC_00012.12.

By LAG3 protein” is meant a protein, a polypeptide, or a fragment thereof having at least about 90% amino acid identity to a LAG 3 gene. An example of a Homo sapiens LAG3 protein having NCBI Reference Sequence NP_002277.4 (SEQ ID NO: 1) is shown below:

| | |
|---|---|
| 1 | mweaqflgll flqplwvapv kplqpgaevp vvwaqegapa qlpcsptipl qdLsLlrrag |
| 61 | vtwqhqpdsg ppaaapghpl apgphpaaps swgprprryt vlsvgpgglr sgrlplqprv |
| 121 | qldergrqrg dfslwlrpar radageyraa vhlrdralsc rlrlrlgqas mtasppgslr |
| 181 | asdwvilncs fsrpdrpasv hwfrnrgqgr vpvresphhh laesflflpq vspmdsgpwg |
| 241 | ciltyrdgfn vsimynltvl glepptpltv yagagsrvgl pcrlpagvgt rsfltakwtp |
| 301 | pgggpdllvt gdngdftlrl edvsqaqagt ytchihlqeq qlnatvtlai itvtpksfgs |
| 361 | pgslgkllce vtpvsgqerf vwssldtpsq rsfsgpwlea qeaqllsqpw qcqlyggerl |
| 421 | lgaavyftel sspgaqrsgr apgalpaghl llflilgvls llllvtgafg fhlwrrqwrp |
| 481 | rrfsaleqgi hppqaqskie eleqepepep epepepepep epeql |

By “α-synuclein gene” is meant a nucleic acid sequence able to express an α-synuclein protein, a polypeptide, or a fragment thereof including the human DNA sequence at the NCBI Gene ID 6622.

By “α-synuclein protein” is meant a protein, a polypeptide, or a fragment thereof having at least about 90% amino acid identity to a α-synuclein gene. An example of an Rattus norvegicus α-synuclein protein is the sequence at NCBI GenBank Number AAS55695.1 (SEQ ID NO: 2) is shown below:

| | |
|---|---|
| 1 | mdvfmkglsk akegvvaaae ktkqgvaeaa gktkegvlyv gsktkegvvh gvttvaektk |
| 61 | eqvtnvggav vtgvtavaqk tvegagniaa atgfvkkdqm gkgeegypqe giledmpvdp |
| 121 | sseayempse egyqdyepea |

By “LAG3 antibody” is meant an antibody that selectively binds a LAG3, preferably at the LAG3 α-syn PFF receptor binding site.

By “anti-α-syn PFF antibody” is meant an antibody that selectively binds a α-syn PFF.

By “disease” is meant any condition or disorder that damages or interferes with the normal function of a cell, tissue, or organ. Examples of diseases include pancreatic cancer.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

“Diagnostic” means identifying the presence or nature of a pathologic condition, i.e., pancreatic cancer. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

By “marker” is meant any protein or polynucleotide having an alteration in expression level or activity that is associated with a disease or disorder. The term “biomarker” is used interchangeably with the term “marker.”

The term “measuring” means methods which include detecting the presence or absence of marker(s) in the sample, quantifying the amount of marker(s) in the sample, and/or qualifying the type of biomarker. Measuring can be accomplished by methods known in the art and those further described herein, including but not limited to immunoassay. Any suitable methods can be used to detect and measure one or more of the markers described herein. These methods include, without limitation, ELISA and bead-based immunoassays (e.g., monoplexed or multiplexed bead-based immunoassays, magnetic bead-based immunoassays).

As used herein, “obtaining” as in “obtaining an agent” includes synthesizing, purchasing, or otherwise acquiring the agent.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

“Immunoassay” is an assay that uses an antibody to specifically bind an antigen (e.g., a marker). The immunoassay is characterized by the use of specific binding properties of a particular antibody to isolate, target, and/or quantify the antigen.

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind, for example, a α-syn PFF receptor site such as LAG3 or to α-syn PFF. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

By “specifically binds” is meant a compound or antibody that recognizes and binds a polypeptide of the invention, but which does not substantially recognize and bind other molecules in a sample, for example, a biological sample, which naturally includes a polypeptide of the invention.

As used herein, the term “sensitivity” is the percentage of subjects with a particular disease.

As used herein, the term “specificity” is the percentage of subjects correctly identified as having a particular disease i.e., normal or healthy subjects. For example, the specificity is calculated as the number of subjects with a particular disease as compared to non-cancer subjects (e.g., normal healthy subjects).

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 mug/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Such treatment (surgery and/or chemotherapy) will be suitably administered to subjects, particularly humans, suffering from, having, susceptible to, or at risk for pancreatic cancer or disease, disorder, or symptom thereof. Determination of those subjects “at risk” can be made by any objective or subjective determination by a diagnostic test or opinion of a subject or health care provider (e.g., genetic test, enzyme or protein marker, a marker (as defined herein), family history, and the like). In particular embodiments, determination of subjects susceptible to or having a pancreatic cancer is determined by measuring levels of at least one of the markers of the invention (e.g., CA19-9, MIA, MIC-1, CEACAM-1, OPN, SPON1, HSP27, POSTN, or LGALS3BP). In particular embodiments, a subject determined susceptible to or having a pancreatic cancer is selected for surgery.

The term “activity” refers to the ability of a gene to perform its function such as ZnT8 (a zinc transporter) being able to transport zinc.

The term “express” refers to the ability of a gene to express the gene product including for example its corresponding mRNA or protein sequence (s).

The term “reference” refers to a standard or control conditions such as a sample (human cells) or preoteolipisomes with a zinc transporter ZnT8 free, or substantially free, of agent.

As used herein, the term “subject” is intended to refer to any individual or patient to which the method described herein is performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

### BRIEF DESCRIPTION OF THE DRAWINGS

FIG. 1A-1E illustrates α-Syn PFF binds to LAG3. (A) Individual clones from a library consisting of 352 individual cDNAs encoding transmembrane proteins (GFC-transfection array panel, Origene) were transfected into SH-SY5Y cells, and the relative binding signals of human α-syn PFF to individual transmembrane proteins are shown. Positive candidates are LAG3 (NM_002286), NRNX1 (NM_138735) and APLP1 (NM_005166). (B) Mouse α-syn-biotin monomer and α-syn-biotin PFF binding affinity to SH-SY5Y cells expressing the indicated proteins. LAG3* Kd assessment was performed without Triton X-100. All other experiments were performed with 0.1% Triton X-100. Transmembrane proteins similar to the candidates were also tested. Quantification of bound α-syn-biotin PFF to the candidates was performed with ImageJ. Kd values are means±SEM and are based on monomer equivalent concentrations. Selectivity was calculated by dividing Kd (monomer) by Kd (PFF). Binding of α-syn-biotin monomer was detected at a concentration of 3000 nM, but binding was not saturable. (C) α-Syn-biotin monomer or α-syn-biotin PFF binding to LAG3-overexpressing SH-SY5Y cells as a function of total α-syn concentration in 0% Triton X-100 (TX-100) or 0.1% TX-100 conditions (monomer equivalent for PFF preparations, top panel). Scatchard analysis (bottom panel). Kd=71 nM (TX-100) and 77 nM (TX-100), data are the means±SEM, n=3. (D) Binding of α-syn-biotin PFF to cultured cortical neurons (21 days in vitro (DIV)) is reduced by LAG3 knockout (LAG3−/−), as assessed by alkaline phosphatase assay. α-Syn-biotin PFF WTKd=374 nM, LAG3−/−−Kd=449 nM, estimated Kd for neuronal LAG3 [dashed line: ΔLAG3=wild-type (WT) minus LAG3−/−] is 103 nM. Data are the means±SEM, n=3. * P<0.05, Student's t-test. Power (1-β err prob)=1. (E) Specificity of LAG3 binding with α-syn-biotin PFF (red box in Fig. S4A). Tau-biotin PFF (green box in Fig. S8A) and β-amyloid-biotin oligomer (blue box in Fig. S9A) are negative controls.

FIG. 2A-2D illustrates endocytosis of α-syn PFF is dependent on LAG3. (A) Live image analysis of the endocytosis of α-syn-pHrodo PFF. α-Syn PFF was conjugated with a pH dependent dye (pHrodo red), in which fluorescence increases as pH decreases from neutral to acidic environments. White triangles indicate non-transfected wild-type (WT) or LAG3−/−neurons and white arrows indicate LAG3 transfected neurons. Scale bar, 10 μm. (B) Quantification of panel A, cell number (5-46) from n=3. (C) Internalized α-syn-biotin PFF co-localizes with Rab5. Co-localization of internalized α-syn-biotin PFF and Rab5 was assessed by confocal microscopy, scale bar, 10 μm. (D) Quantification of panel C, cell number (13-32) from n=4. One-way ANOVA with Tukey's correction. Data in B and D are as means±SEM. *P<0.05, **P<0.01, ***P<0.001. Power (1-β err prob)=1.

FIG. 3A-3I illustrates α-Syn PFF induced pathology and transmission is reduced by deletion of LAG3 in vitro. (A) WT and LAG3−/− primary cortical neurons at 7 DIV were treated with α-syn PFF or PBS. LAG3 was overexpressed via Lenti-virus (LV) transduction in WT or LAG3−/− neurons at 4 DIV. 3 days after transducton, 7 DIV cultures were treated with α-syn PFF or PBS. All the cultures were fixed 10-day post-treatment in 4% PFA. Neurons were stained with rabbit mAb MJF-R13 (8-8) for P-α-syn. Scale bar, 40 μm. (B) Quantification of panel A, n=5 independent experiments, each performed in duplicate. Values are given as the means±SEM. Statistical significance was determined using one-way ANOVA followed with Tukey's correction, ***P<0.001. Power (1-β err prob)=1. (C-G) Immunoblots of misfolded α-syn and P-α-syn levels in WT and LAG3−/− neuron lysates sequentially extracted in 1% TX-100 (TX-soluble) followed by 2% SDS (TXinsoluble) 14 days after PFF treatment. α-Syn PFF recruited endogenous α-Syn into TXinsoluble and hyperphosphorylated aggregates, which was ameliorated by deletion of LAG3. α-Syn PFF caused a reduction in levels of SNAP25 and synapsin II compared to PBS 14 days post-treatment. Deletion of LAG3 prevented PFF-induced synaptic protein loss. Values are given as means±SEM, n=3 independent experiments. Statistical significance was determine using one-way ANOVA followed by Tukey's correction, *P<0.05, **P<0.01, ***P<0.001. (H) Deletion of LAG3 prevents transmission of pathological α-syn aggregates. Schematic of microfluidic neuron device with three chambers to separate neurons seeded in three chambers. Transmission of pathologic P-α-syn from chamber 1 (C1) to chamber 2 (C2) to chamber 3 (C3) 14 days post-addition of α-syn PFF in C1. The different combinations of neurons tested in C2, listed as C1-(C2)-C3, are: WT−(WT)−WT, WT−(WT+LAG3)−WT, WT−(LAG3−/−)−WT, WT−(LAG3−/−+LAG3)−WT. Scale bar, 10 μm. (G) Quantification of panel F. Values are given as means±SEM, n=3. Statistical significance was determine using one-way ANOVA followed by Tukey's correction, *P<0.05, **P<0.01, ***P<0.001. Power (1-β err prob)=1. (I) Graph of P-α-syn levels.

FIG. 4A-4E illustrates α-Syn PFF induced pathology is reduced by deletion of LAG3 in vivo. (A) Representative P-α-syn immunostaining and quantification in the substantia nigra par compacta (SNpc) of WT and LAG3−/− mice sacrificed at 30 and 180 days after intrastriatal α-syn PFF injection. Data are the means±SEM, n=5-9 mice per group, one-way ANOVA with Sidak's correction. (B) Stereology counts from TH immunostaining and Nissl staining of SNpc DA neurons of WT and LAG3−/− mice at 180 days after intrastriatal α-syn PFF, α-syn monomer or PBS injection. Data are the mean number of cells per region±SEM, n=5-9 mice per group, one-way ANOVA with Dunnett's correction. (C) DA concentrations in the striatum of α-syn PFF-injected mice and PBS-treated controls measured at 180 days by HPLC. Data are the means±SEM, n=5-8 mice per group, one-way ANOVA with Tukey's correction. (D, E) 180 days after α-syn PFF injection, the pole test and grip strength was performed in WT or LAG3−/− mice injected with PBS or α-syn PFF. Behavioral abnormalities in the pole test and grip strength induced by α-syn PFF injection were ameliorated in LAG3−/− mice. Data are the means±SEM, n=7-9 mice per group for behavioral studies. Statistical significance was determined using one way ANOVA with Tukey's correction, * P<0.05, *** P<0.001, n.s., nonsignificant. Power (1-β err prob)=1.

FIG. 5A-5D illustrates α-Syn-biotin labeled monomer and PFF. (A) α-Syn-biotin labeled monomer and α-syn-biotin PFF was analyzed using size exclusion chromatography (SEC) via fast protein liquid chromatography (FPLC) by monitoring the peak absorbance at 280 nm (red). Peak ‘a’ is α-syn-biotin PFF (>50 monomers) and ‘b’ is α-syn-biotin monomer (B) Recombinant α-syn-biotin labeled monomer and α-syn-biotin PFF were validated by immunoblot using an anti-α-syn antibody (BD Biosciences). The migration of molecular mass markers (kDa) is indicated on the left. (* bottom of the gel). (C) α-Syn-biotin labeled monomer and α-syn-biotin PFF were examined by atomic force microscopy (AFM). Scale bar, 300 nm. (D) α-syn-biotin labeled monomer and α-syn-biotin PFF were characterized by transmission electron microscopy (TEM). Scale bar, 100 nm.

FIG. 6A-6C illustrates α-Syn-biotin PFF binds to wild-type mouse primary cortical neuron. (A) α-Syn-biotin PFF binds to neuron in a saturable manner, as a function of α-syn-biotin total concentration (monomer equivalent for PFF preparations). Scale bar, 100 μm. (B) High magnification views of images in panel A of α-syn-biotin PFF binding on neurons. Scale bar, 20 μm. (C) α-Syn-biotin PFF K_{d}=374 nM, α-syn-biotin monomer K_{d}=2734 nM. Data are the means±SEM, n=3.

FIG. 7A-7C illustrates the screening strategy for α-syn PFF binding proteins. (A) Schematic diagram outlining the strategy for screening of α-syn-biotin PFF binding proteins. The details of the screening are explained in the materials and method section. (B-C) Screening and selection of a cell line with low background binding for α-syn-biotin PFF. SH-SY5Y cells exhibit<8% of the α-syn-biotin PFF binding level compared to wild-type primary cortical neurons. Data are the means±SEM, n=3. Scale bar, 10 μm. μm. (C) Human α-syn PFF binding to human recombinant LAG3 by ELISA assay. Kd=2.7 nM.

FIG. 8A-8B illustrates LAG3 binds to α-syn-biotin PFF but not α-syn-biotin monomer. (A) Comparison of α-syn-biotin monomer and α-syn-biotin PFF binding to LAG3-expressing SH-SY5Y cells and to CD4-expressing SH-SY5Y cells. Scale bar. 100 μm. The binding experiments for LAG3 includes 0% Triton X-100 (TX-100) and 0.1% TX-100. (B) High magnification images of panel A demonstrating α-syn-biotin PFF binding. Scale bar, 20 μm.

FIG. 9A-9B illustrates LAG3 expression and localization. (A) LAG3 expression in wild-type (WT) cortical cultures, HEK293FT cells. SH-SY5Y cells and LAG3^{-/-} cortical cultures. β-actin is provided as a loading control. (B) LAG3 expression in WT cortical neurons (TUJ1), WT astrocytes (GFAP) and WT microglia (Iba-1). β-actin is provided as a loading control. *non-specific band. Immunoblots in separate experiments were replicated three times and show similar results.

FIG. 10A-10B illustrates a comparison of α-syn-biotin PFF to wild-type (WT) and LAG3^{-/-} mouse primary cortical neurons. (A) α-Syn-biotin PFF binding to wild-type (WT) from fig. S2 is shown and compared to α-syn-biotin PFF binding to LAG3^{-/-} neurons. Scale bar, 100 μm. (B) High magnification views of images in panel A of α-syn-biotin PFF binding on WT and LAG3^{-/-} neurons. Scale bar, 20 μm.

FIG. 11A-11D illustrates pathological α-syn binds to LAG3. (A) α-Syn-biotin PFF binds to LAG3 as determined by streptavidin beads that pull down of α-syn-biotin PFF, while α-syn-biotin monomer does not bind in HEK293 FT cells transfected with GFP and LAG3. n=3 independent experiments. (B) LAG3 binds α-syn-biotin PFF as detected by an anti-LAG3 antibody (410C9) pull down, while α-syn-biotin monomer does not bind to α-syn-biotin PFF. n=3 independent experiments. (C) LAG3 binds to aggregated α-syn in vivo from the brain stem of 10 month old human A53T α-syn transgenic mice, while monomeric α-syn from brain stem of 4 month old human A53T α-syn transgenic mice and 4 and 10 month old wild-type (WT) does not bind to LAG3. n=3 independent experiments. (D) Human α-syn PFF binds to human recombinant LAG3 as assessed by ELISA. K_{d}=2.7 nM, n=3.

FIG. 12A-12B illustrates Tau PFF does not bind to LAG3. (A) Low power images of Tau-biotin PFF binding to non-transfected and LAG3 transfected SH-SY5Y cells. (B) High magnification views of images in panel A of Tau-biotin PFF binding on non-transfected and LAG3 transfected SH-SY5Y cells. Scale bar, 20 μm. Tau-biotin PFF binds to non-transfected cells in a saturable manner while overexpression of LAG3 fails to increase the binding of tau-biotin PFF. Experiments were replicated three times and show similar results.

FIG. 13A-13B illustrates β-amyloid oligomer and β-amyloid PFF does not bind to LAG3. (A) Low power images of 3-amyloid-biotin oligomer and PFF binding to non-transfected and LAG3 transfected SH-SY5Y cells. (B) High magnification views of images in panel A of β-amyloid-biotin oligomer and β-amyloid-biotin PFF on non-transfected and LAG3 transfected SH-SY5Y cells. Scale bar. 20 μm. β-Amyloid-biotin oligomer and β-amyloid-biotin PFF bind to both non-transfected and LAG3 overexpressing SH-SY5Y cells at high concentrations in a non-specific manner. Experiments were replicated three times and show similar results.

FIG. 14A-14B illustrates mapping of the domain of LAG3 that binds α-syn PFF. (A) Schematic diagram of mouse LAG3 domains and deletions mutants. LAG3 ectodomain is composed of four Ig-like domains (D1-D4). The D1 domain was divided into five deletion mutants (del1-5-D1). HEK293FT cells were transfected with expression plasmids directing the expression of each of the indicated LAG3 deletion mutants. Transfected cells were assessed for binding of α-syn-biotin PFF. (B) Top panel, binding images of α-syn-biotin PFF to full-length (FL) and deletion mutants of LAG3: extracellular domains (ΔD1-ΔD4), intracellular domain (ΔICD), and subdomains of D1 domain (Δdel1-5-D1). Bottom panel, quantification of top panel. Scale bar, 100 μm and 10 μm. Data are the means±SEM, n=5-8, **P<0.01, *** P<0.001 compared to FL, one-way ANOVA followed by Dunnett's correction. Power (1-β err prob)=1.

FIG. 15A-15F illustrates endocytosis of α-syn PFF. (A) α-Syn PFF is conjugated with pHrodo red dye, which is non-fluorescent at pH7 (mimicking the extracellular pH environment) (left panel), but fluoresces brightly at pH 4 (mimicking the lysosomal pH environment) (middle panel). Conjugation of pHrodo red to α-syn PFF does not alter the band analyzed by immunoblot (right panel). (* bottom of the gel) (B) The fibrillar structure of α-syn-pHrodo PFF is shown by AFM, scale bar, 200 nm. (C) Enlarged live images of FIG. 2A showing the endocytosis of α-syn-pHrodo PFF in wild-type (WT), WT+LAG3, LAG3^{-/-}, LAG3^{-/-}+LAG3 groups. Scale bar, 10 μm. White square highlights the image shown in FIG. 2A. (D) Live images of the endocytosis of α-syn-pHrodo PFF in WT and LAG3^{-/-} neurons marked by AAV2-eSYN-EGFP-WPRE. Scale bar, 10 μm. (E) Live images of the endocytosis of α-syn-pHrodo PFF in the setting of expression of deletion mutants of LAG3 in LAG3^{-/-} primary neuron cultures. (F) Quantification of panel E, cell number (5) from n=3. Data are the means±SEM. *P<0.05,**P<0.01 compared to full length LAG3, One way ANOVA with Tukey's correction. Scale bar, 10 μm.

FIG. 16A-16D illustrates co-localization of α-syn-biotin PFF with Rab5, Rab7 and LAMP1. (A) Co-localization of LAG3 (blue), Rab5 (green) and α-syn-biotin PFF (red) in cortical neurons, Scale bar, 10 μm. High magnification images of the white boxes are shown below each image along with the Y- and Z-planes. Arrow highlights the co-localization. (B) Co-localization of α-syn-biotin PFF (red) Rab5 (green) and LAG3 (grey scale) in dendrites of wild-type (WT) and LAG3^{-/-} neuronal cultures. Scale bar, 10 μm. (C) Quantification of α-syn-biotin PFF colocalization with Rab5, n=3. Data are as means±SEM. * P<0.05, *** P<0.001. One way-ANOVA with Tukey's correction. (D) Co-localization of α-syn-biotin PFF (red) with the endosome markers, Rab7 (green) and LAMP1 (green). Y- and Z-planes are shown in the merged images. Scale bar, 10 μm. Experiments were replicated three times and show similar results.

### DETAILED DESCRIPTION OF THE INVENTION

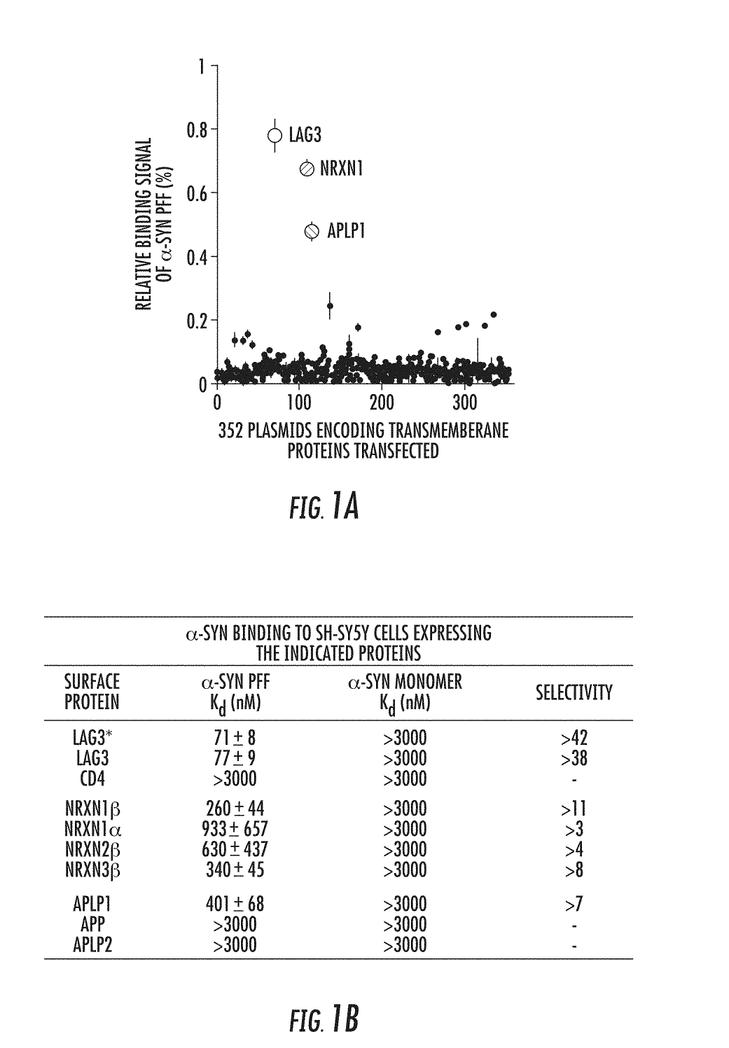

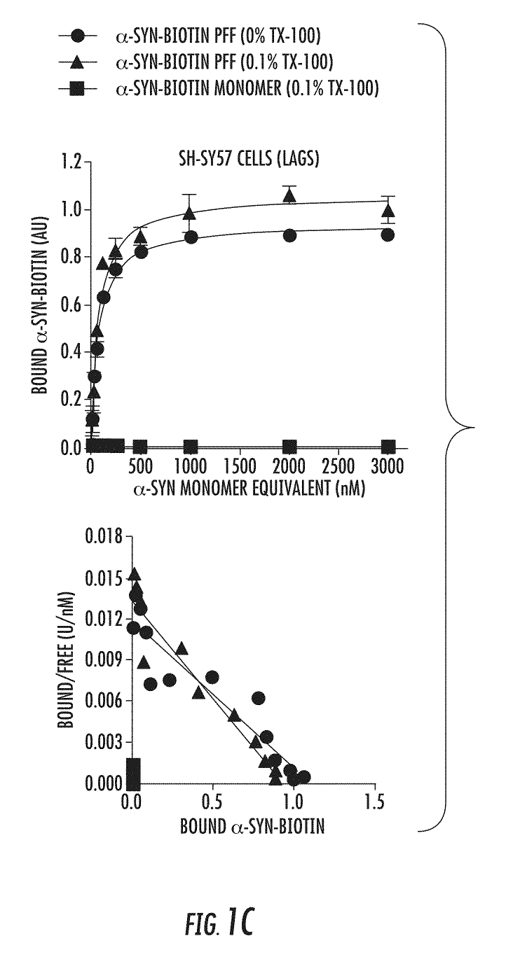

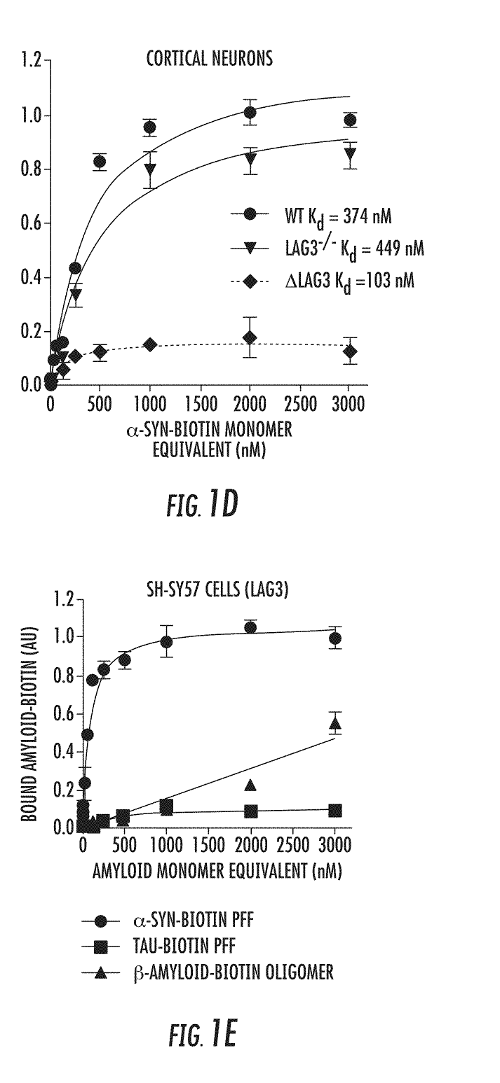

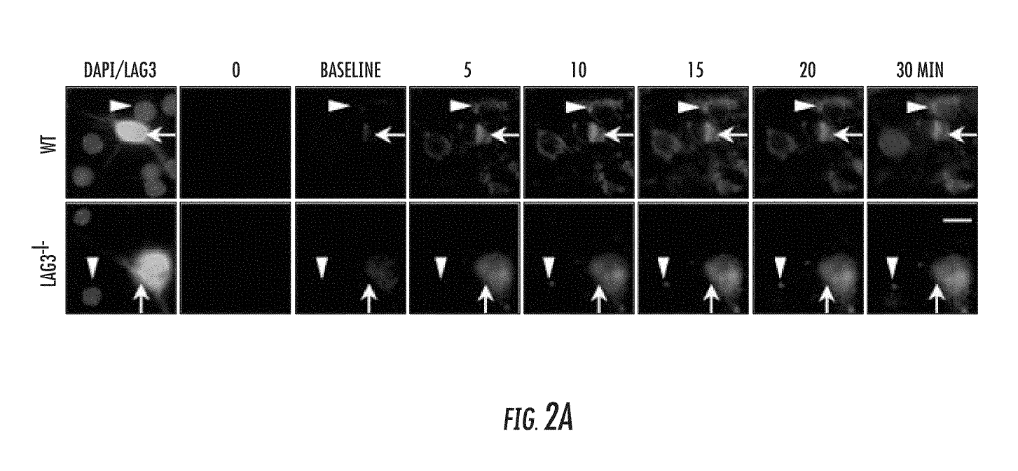

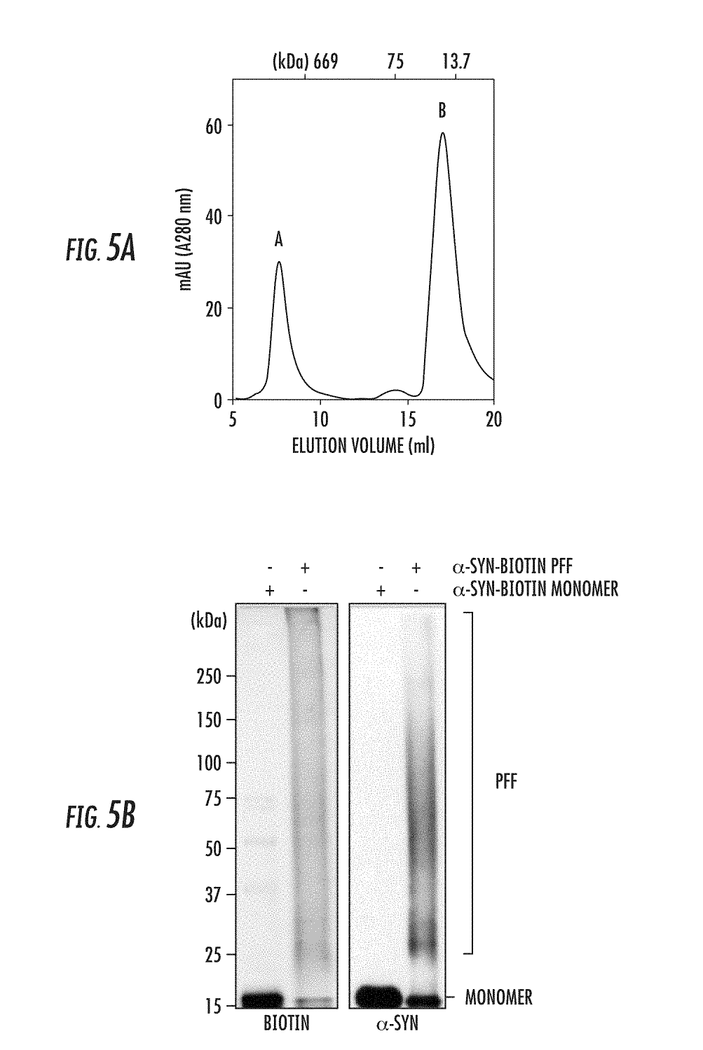

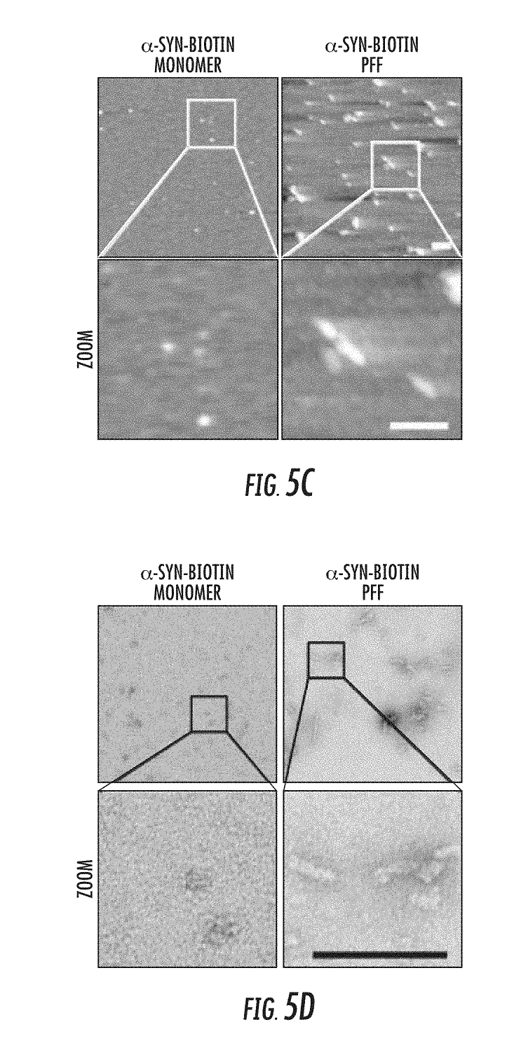

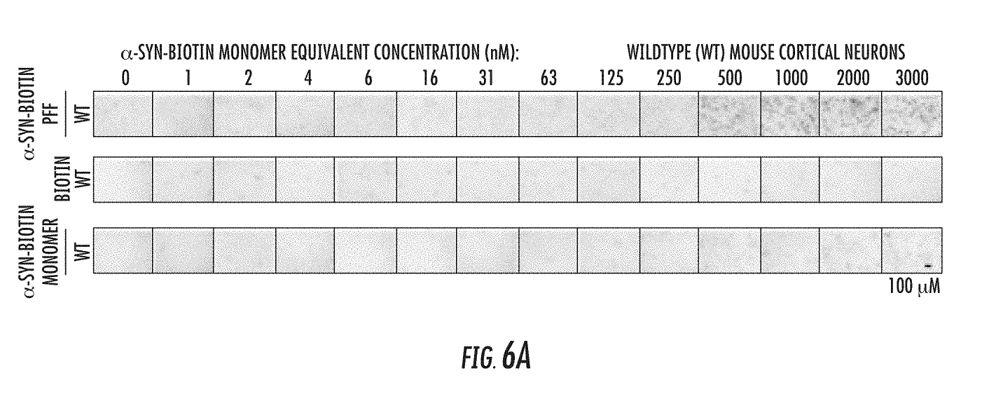

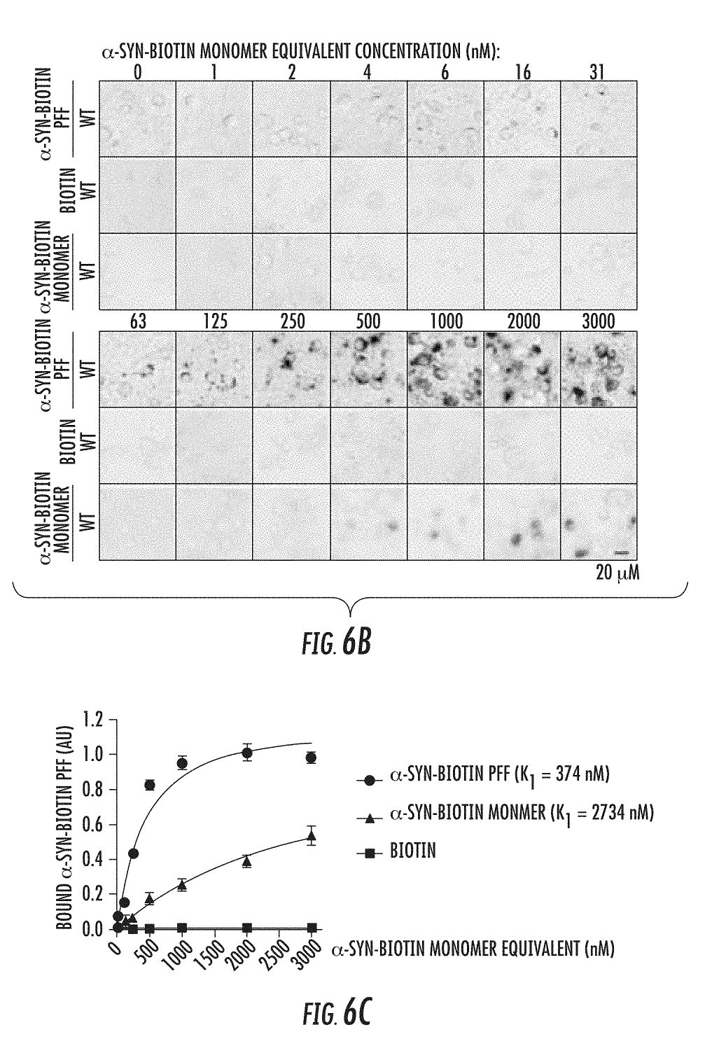

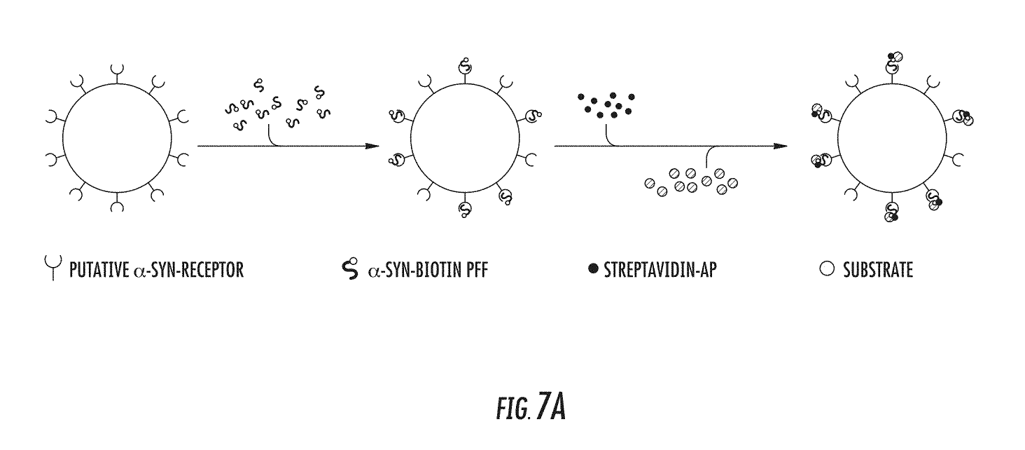

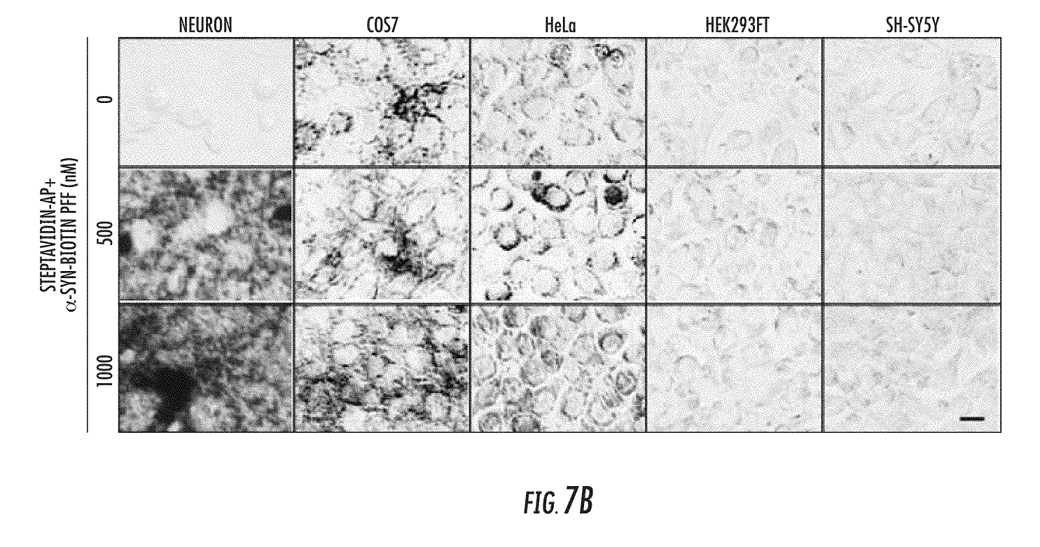

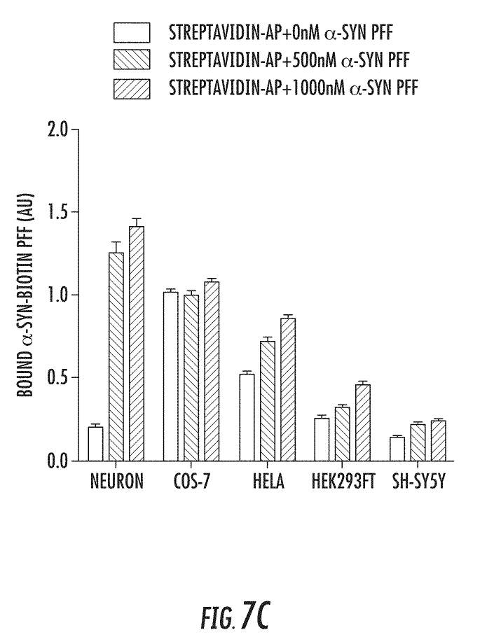

α-Syn was synthesized and conjugated to biotin (α-syn-biotin), and then aggregated over seven days followed by sonication to form PFF. Size exclusion chromatography was used to separate PFF from α-syn monomers (FIG. 5A). Recombinant α-syn-biotin monomers and PFF were validated by immunoblot analysis (FIG. 5B). α-syn-biotin monomers and PFF were examined by atomic force microscopy. α-syn-biotin monomers exhibit no regular structure whereas α-syn-biotin PFF exhibit short fibrillar structures (FIG. 5C). The inventors then sought to investigate the interaction between extracellular α-syn and neurons. α-syn-biotin PFF bind to cortical neurons as detected by streptavidin-AP (alkaline phosphatase) staining, whereas α-syn-biotin monomers weakly bind (FIG. 6A). Binding to neurons is saturable with an apparent disassociation constant (K_{d}) of 309 nM (FIG. 6B), suggesting the existence of a receptor for α-syn PFF. We screened for potential α-syn receptor(s) through expression cloning. A key requirement for expression cloning is the existence of a cell line with low α-syn-biotin PFF background binding. SH-SY5Y cells exhibit less than 8% of the binding levels of α-syn-biotin PFF as compared to cortical neurons, whereas COS7 and HeLa cells exhibit relatively high binding, and HEK-293 cells exhibit moderate binding (FIG. 6C). Complimentary DNAs encoding 352 transmembrane proteins (TMGW10001, GFC-transfection array panel, Origene) were expressed in SH-SY5Y cells and screened for α-syn-biotin PFF binding candidates via detection with streptavidin-AP staining (FIG. 6D). Three positive clones were identified that bind α-syn-biotin PFF and include lymphocyte-activation gene 3 (LAG3), Neurexin1β and amyloid beta (A4) precursor-like protein 1 (APLP1) (FIG. 1A). Our screen provides Z-factors of co-efficient of (0.82, 0.84, 0.84) through three independent screenings suggesting that the screen was within the optimal signal window to preclude false positives or negatives.

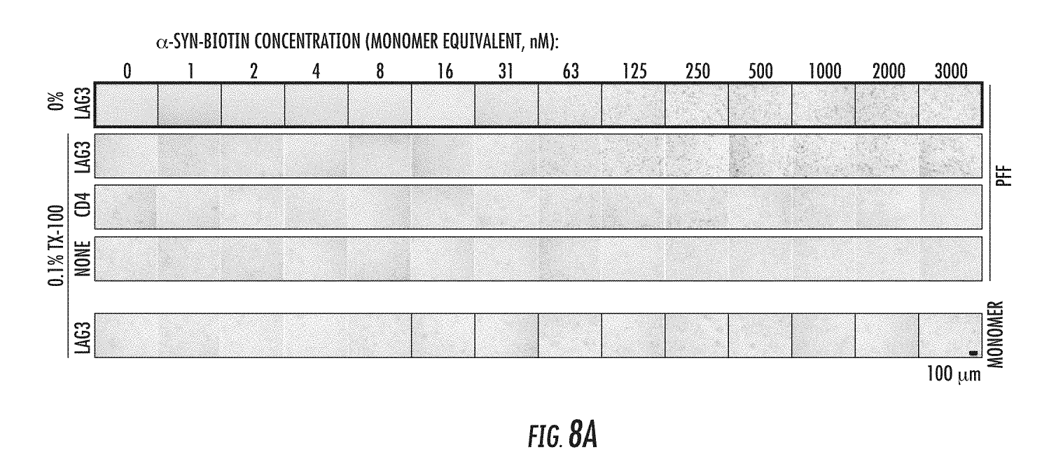

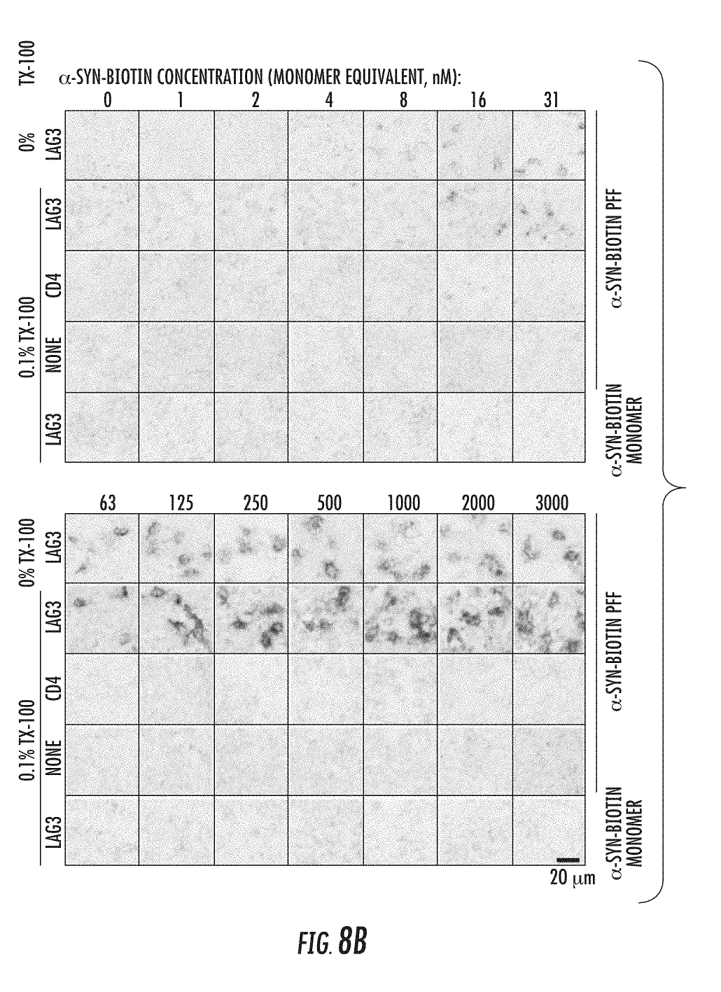

The selectivity of LAG3, Neurexin1β and APLP1 and related transmembrane proteins for α-syn-biotin PFF versus α-syn-biotin monomers was determined via the ratio of K_{d} values (FIG. 1B). LAG3 exhibits the highest selectivity with a ratio of 38 followed by Neurexin1β with a ratio of 11 and APLP1 with a ratio of 7. The binding of α-syn-biotin PFF to LAG3 is specific since α-syn-biotin PFF does not bind to the closely related receptor CD4 (FIG. 1B, FIG. 7A). In addition to α-syn-biotin PFF binding to Neurexin1β, it also binds to Neurexin2β and Neurexin3β and mildly binds to Neurexin1α (FIG. 1B). α-Syn-biotin PFF do not bind amyloid precursor protein (APP) or APLP2 suggesting that the binding to APLP1 is specific (FIG. 1B). Since LAG3 exhibits the highest selectivity for α-syn-biotin PFF, it was advanced for further study. α-Syn-biotin PFF do not exhibit appreciable binding to SH-SY5Y cells alone, but demonstrate substantial binding to LAG3 expressing SH-SY5Y cells, whereas α-syn-biotin monomers do not exhibit appreciable binding (FIGS. 7A-7B). α-Syn-biotin PFF bind to LAG3 in a saturable manner with a K_{d} of 77 nM (FIGS. 1B, 1C, FIG. 7A). α-Syn-biotin monomers do not demonstrate any specific binding to LAG3 expressing SH-SY5Y cells up to 3000 nM (FIGS. 1B, 1C, FIG. 7A). We further confirmed that α-syn-biotin PFF binds to human recombinant LAG3 directly with a K_{d} of 2.7 nM by using an ELISA assay (FIG. 7C). In vitro co-immunoprecipitation (Co-IP) studies show that α-syn-biotin PFF but not α-syn-biotin monomers pull down LAG3 (FIG. 8A) and conversely LAG3 pulls down α-syn-biotin PFF but not α-syn-biotin monomers (FIG. 8B). Moreover, in vivo Co-IP studies show that misfolded α-syn from aged transgenic mice (9) overexpressing human A53T protein pull down LAG3 protein, but not α-syn monomers from young transgenic mice or aged/young wild type mice (FIG. 8C), which suggests that LAG3 binds specifically to pathological species of α-syn. Wild type mouse cortical neurons demonstrate α-syn-biotin PFF binding whereas LAG3 knockout mouse cortical neurons have markedly reduced α-syn-biotin PFF binding (FIG. 1D). Taken together these results indicate the Lag3 is a receptor for α-syn PFF.

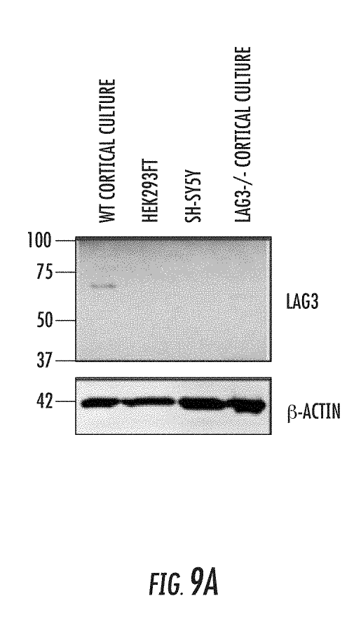

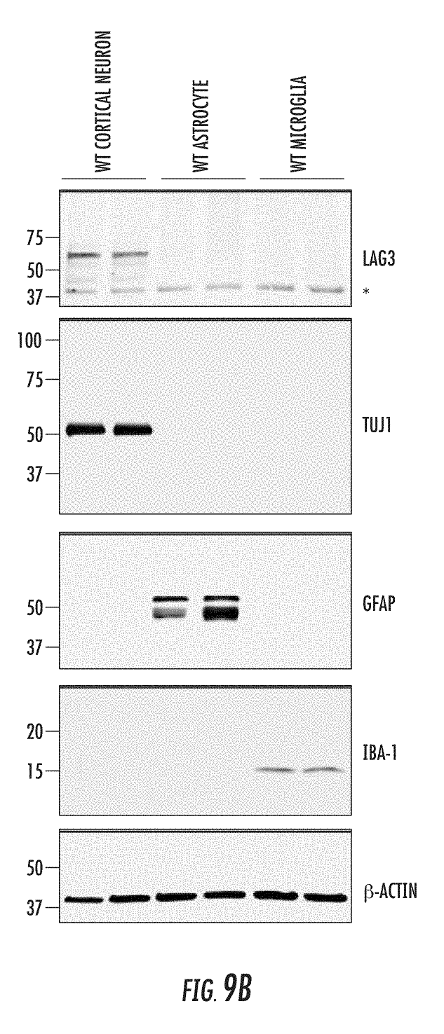

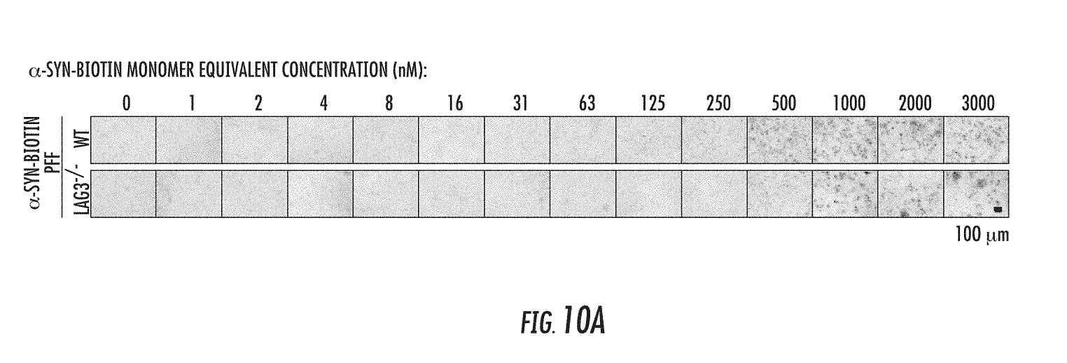

Like other major histocompatibility complex (MHC) class II molecules, LAG3 contains an ectodomain composed of four Ig-like domains (D1-D4). To determine the α-syn-biotin PFF binding domain, we sequentially deleted each domain of LAG3 and performed the cell surface binding assay with overexpression of the LAG3 deletion mutants. These experiments reveal that α-syn-biotin PFF preferentially bind to the D1 domain whereas deletion of the D2, D3 or the intracellular domain (ICD) substantially weakens binding, but not the D4 domain (FIG. 9A-9B). Since α-syn-biotin PFF binding to LAG3 is eliminated by deletion of the D1 domain, additional deletions of D1 subdomains (Δdel1-5) were examined. ΔDel2(aa 52-80)-D1 and Δdel3(aa 81-109)-D1 significantly reduce α-syn-biotin PFF binding to Lag3, while Δdel1(aa23-51)-D1, Δdel4(aa110-138)-D1 and Δdel5(aa139-167)-D1 of D1 moderately reduces binding of LAG3 to α-syn-biotin PFF (FIG. 9A-9B).

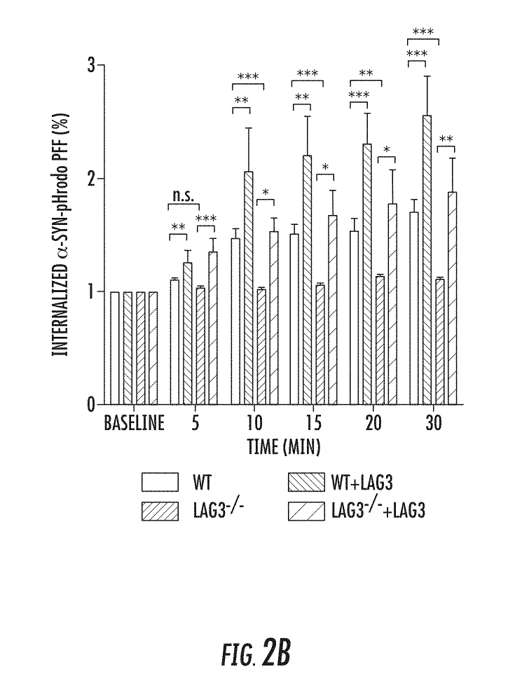

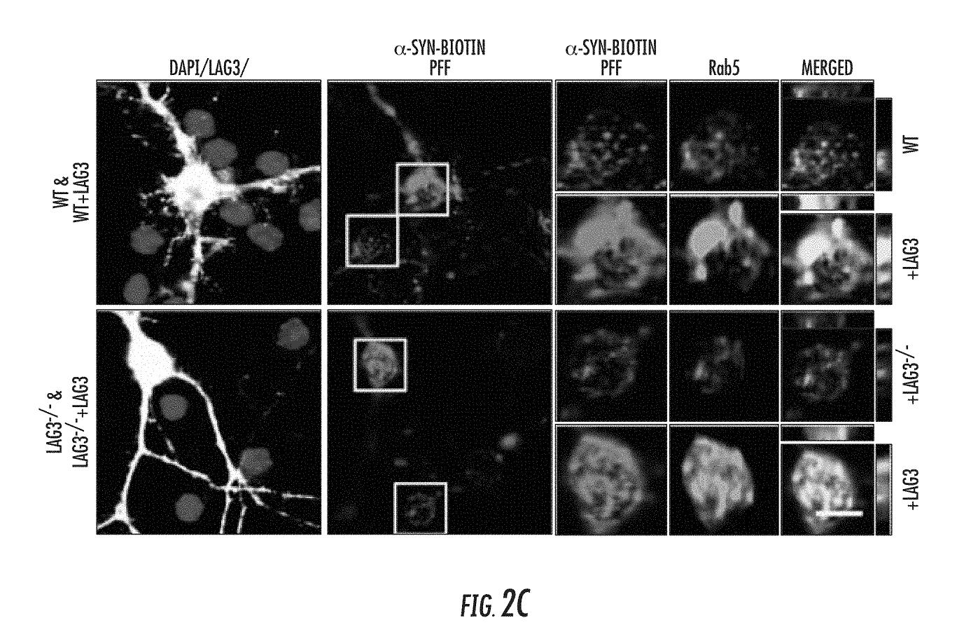

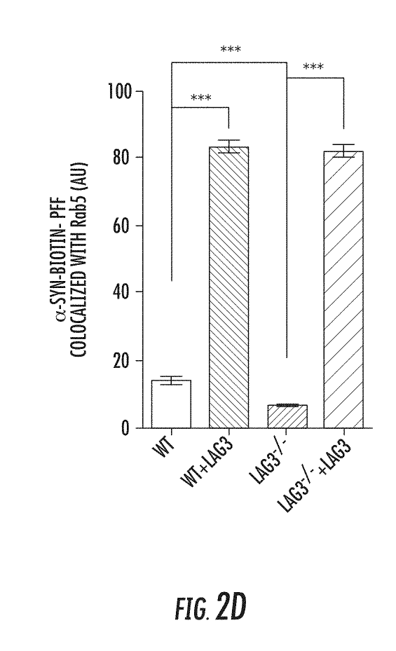

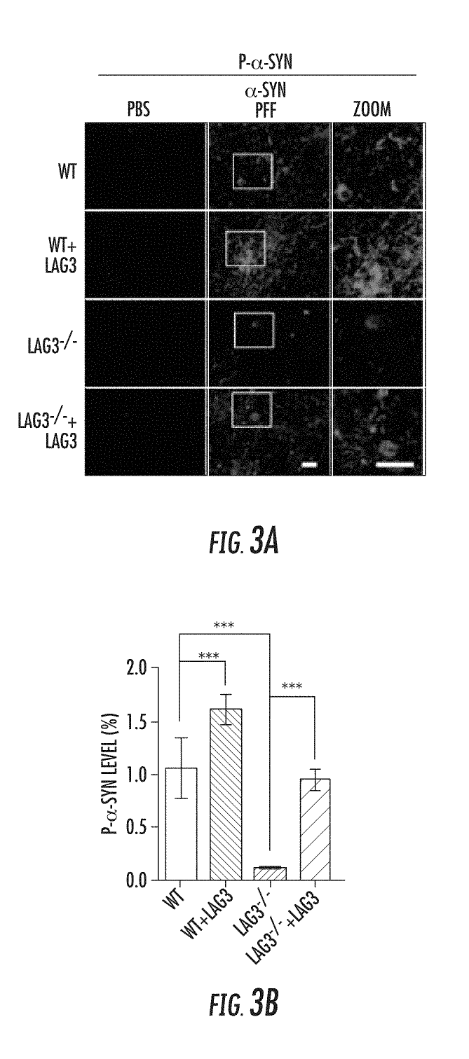

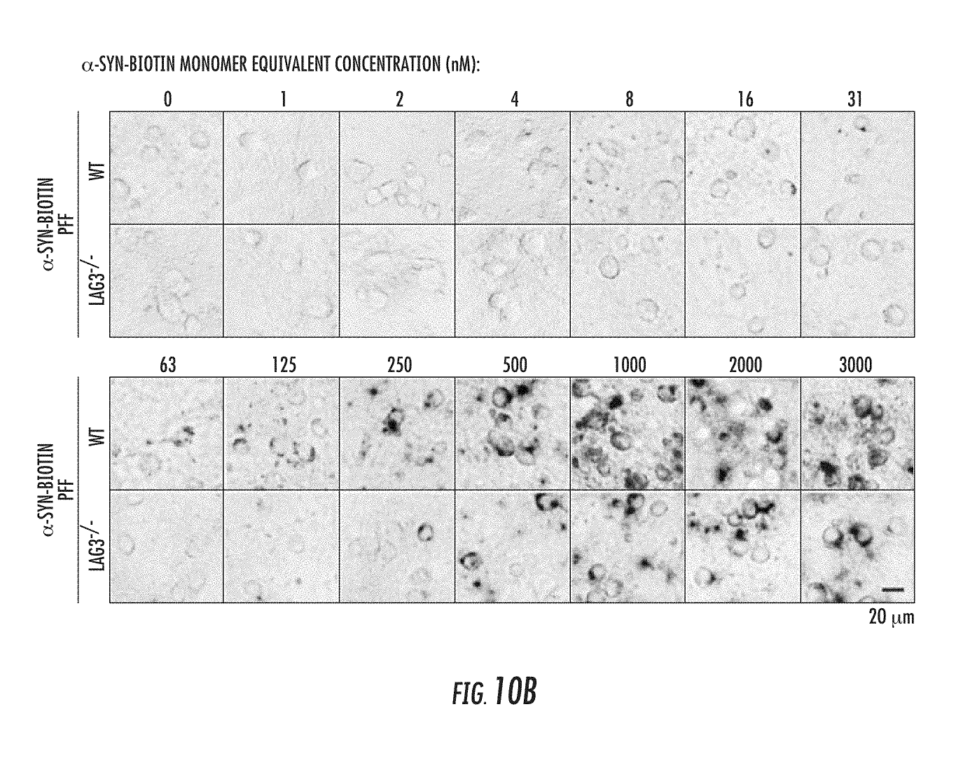

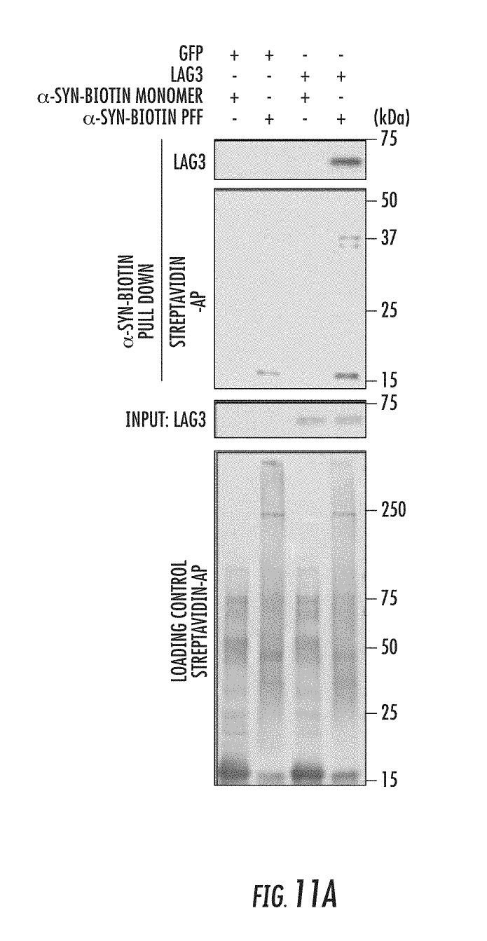

To determine whether LAG3 is required for the endocytosis of α-syn PFF, pHrodo red was conjugated to α-syn PFF. pHrodo red is a pH dependent dye that increases in fluorescence as pH decreases from the neutral cytosolic pH to the acidic pH of the endosome. Conjugation of α-syn PFF with pHrodo red does not appreciably change the properties of the α-syn PFF as assessed by immunoblot and atomic force microscopy (FIG. 10A-10B). α-syn-pHrodo PFF undergoes endocytosis in wild type cortical cultures while LAG3 knockout cultures show essentially no endocytosis (FIG. 2A, 2B, FIG. 10C). Overexpression of LAG3 in wild type culture enhances the endocytosis of α-syn-pHrodo PFF and overexpression of LAG3 in the LAG3 knockout cortical cultures restores endocytosis of α-syn-pHrodo PFF (FIG. 2A, 2B, FIG. 10C). Examination of overexpression of the deletion mutants in LAG3 knockout cortical cultures shows that the D1 domain deletion mutant fails to increase endocytosis of α-syn-pHrodo PFF (FIG. 2A, 2C). Deletions of the D2, D3 or D4 domain D4 domain has no significant effect on endocytosis (FIG. 2A, 2C).

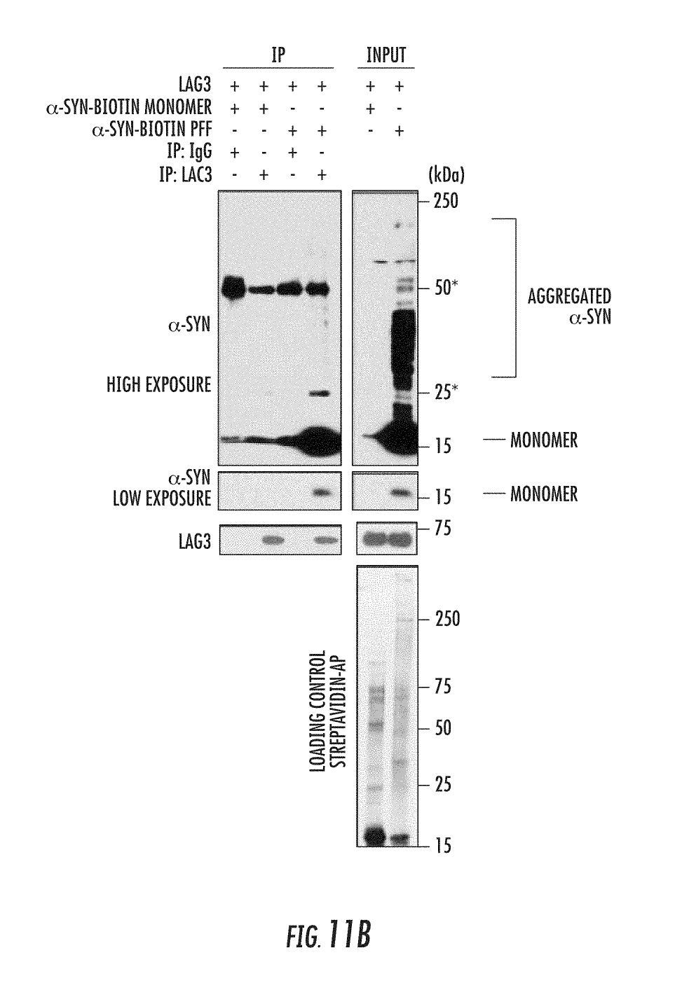

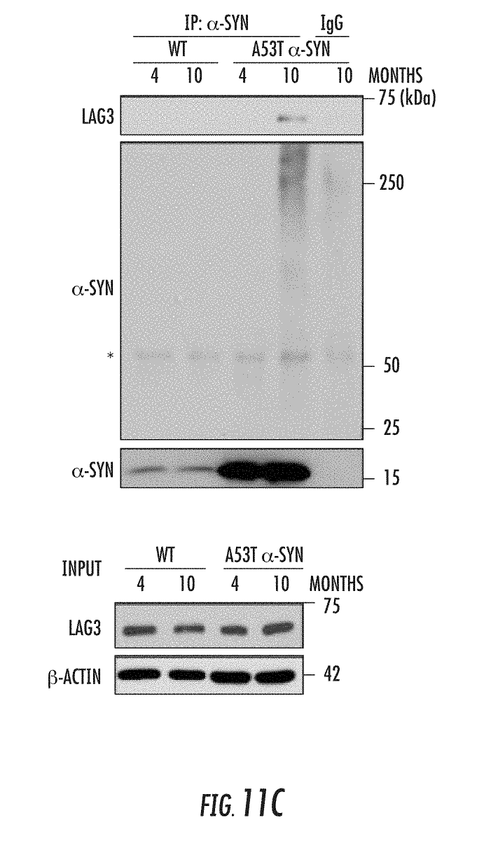

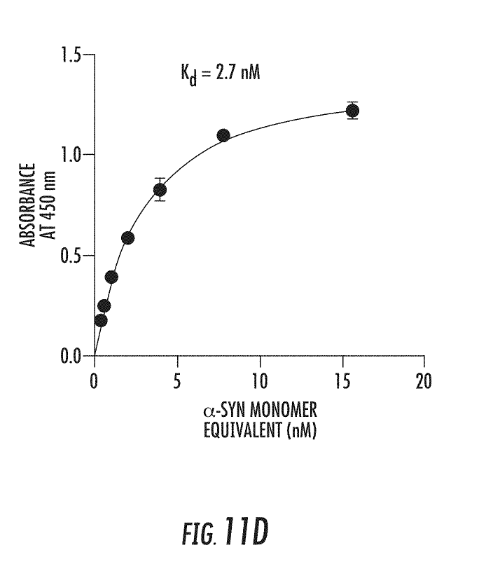

The Rab5 GTPase is an early endosomal marker and helps mediate endocytosis. As such, the inventors sought to confirm the endocytosis of α-syn-biotin PFF into endosomes by measuring the intensity of internalized α-syn-biotin PFF that is co-localized with Rab5. The inventors find that internalized α-syn-biotin PFF is co-localized with Rab5 in wild type cortical neurons (FIG. 2D, 2E). In contrast there is less internalized α-syn-biotin PFF in LAG3 knockout cortical neurons (FIG. 2D, 2E). Overexpression of LAG3 in wild type and LAG3 knockout cortical neurons markedly enhances the internalization of α-syn-biotin PFF (FIG. 2D, 2E, FIG. 11A). LAG3 seems to specifically recognize α-syn PFF, since tau-biotin PFF fail to co-localize with Rab5 in neurons overexpressing LAG3 while α-syn PFF co-localizes with Rab5 in neurons overexpression LAG3 (fig. S7B). Examination of overexpression of the deletion mutants in LAG3 knockout neuronal cultures shows that the D1 domain deletion mutants fail to increase internalization of α-syn-biotin PFF (FIG. 11C-11D). Deletions of the D2, D3, D4 and the intracellular domain (ICD) enhance the internalization α-syn-biotin PFF (fig. S7C-D). Deletions of D1 subdomains (Δdel(1-5)-D1) were also examined for α-syn-biotin PFF endocytosis. Consistent with our binding assays, Δdel2-D1 and Δdel3-D1 have the greatest effect on reducing the endocytosis of α-syn-biotin PFF (fig. S7C-D).

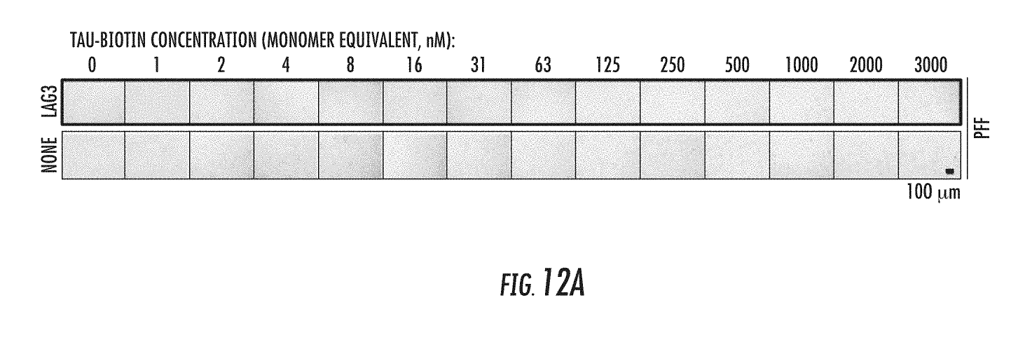

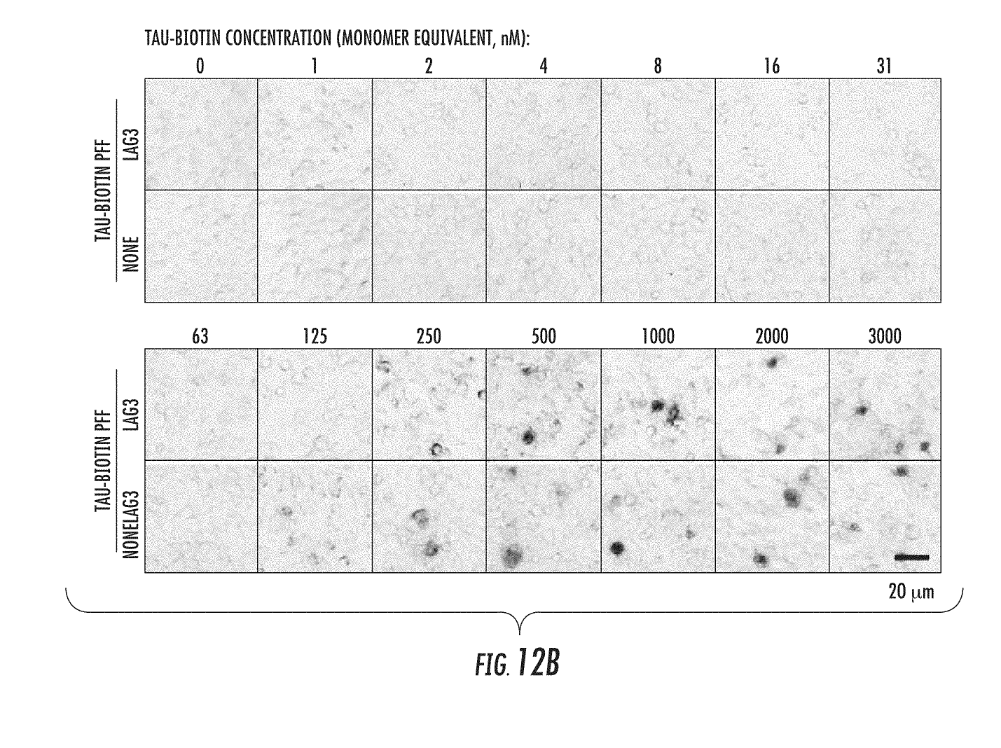

Microsomes, which contain endosomes were isolated via differential centrifugation from wild type and LAG3 knockout cultures following treatment with α-syn-biotin PFF (FIG. 12A). Both monomeric and higher molecular weight forms of α-Syn-biotin PFF are found in the microsome fraction of wild type neuron cultures, while there is significantly less of both forms in LAG3 knockout cultures (FIG. 12B-C). Lenti-viral mediated overexpression of LAG3 in wild type cultures enhances the levels of α-syn-biotin PFF in microsome fractions and restores the levels of α-syn-biotin PFF in LAG3 knockout culture microsome fractions (FIG. 12B-C). Taken together these results indicate that LAG3 is required for the endocytosis of α-syn-biotin PFF into neurons.

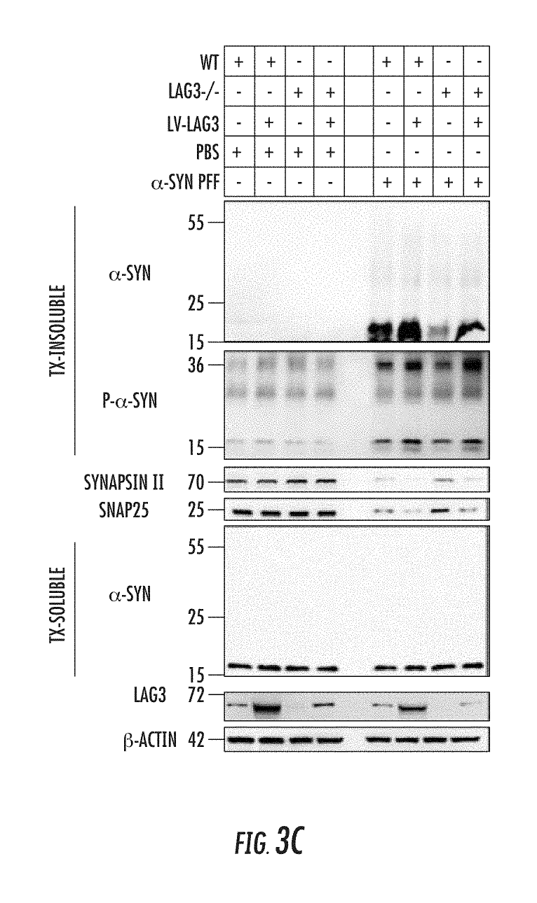

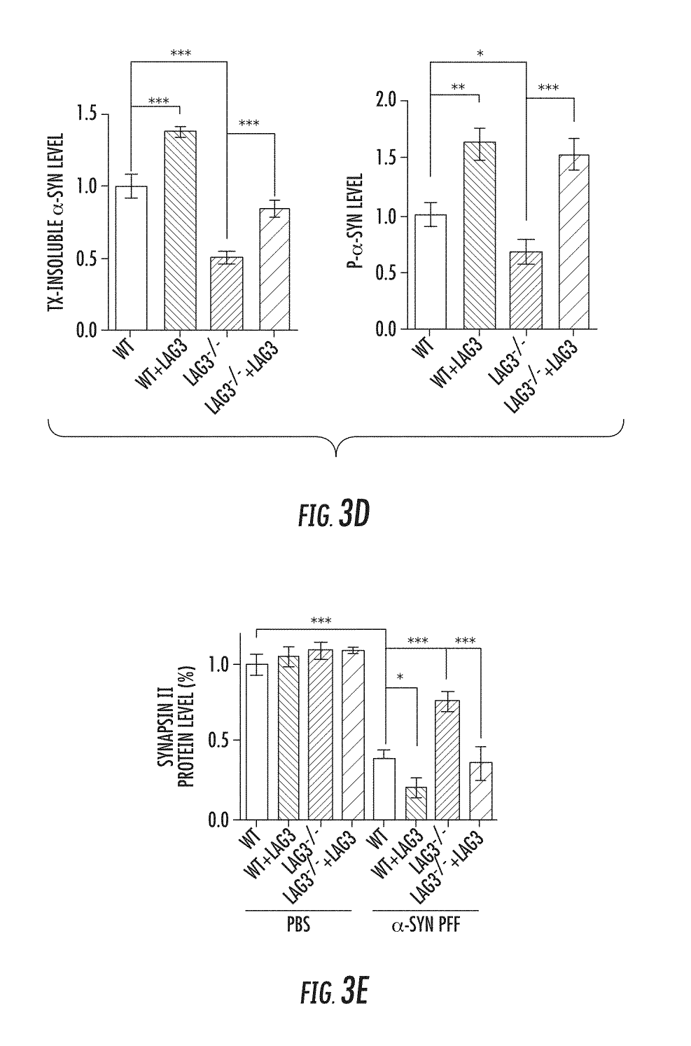

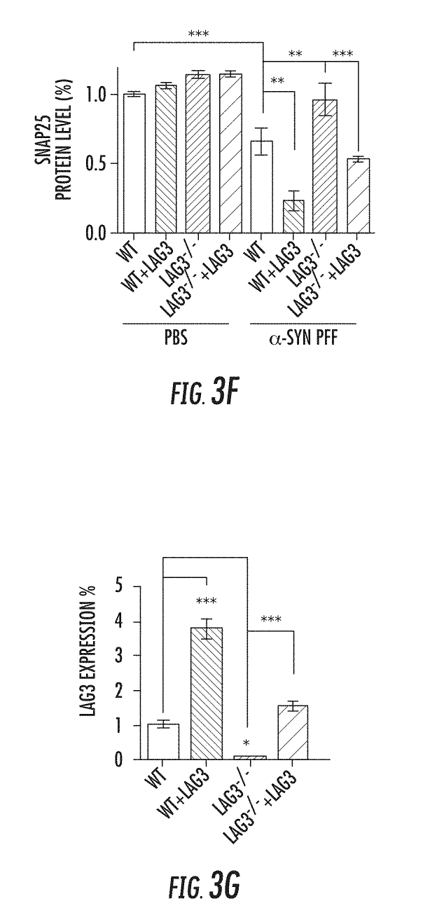

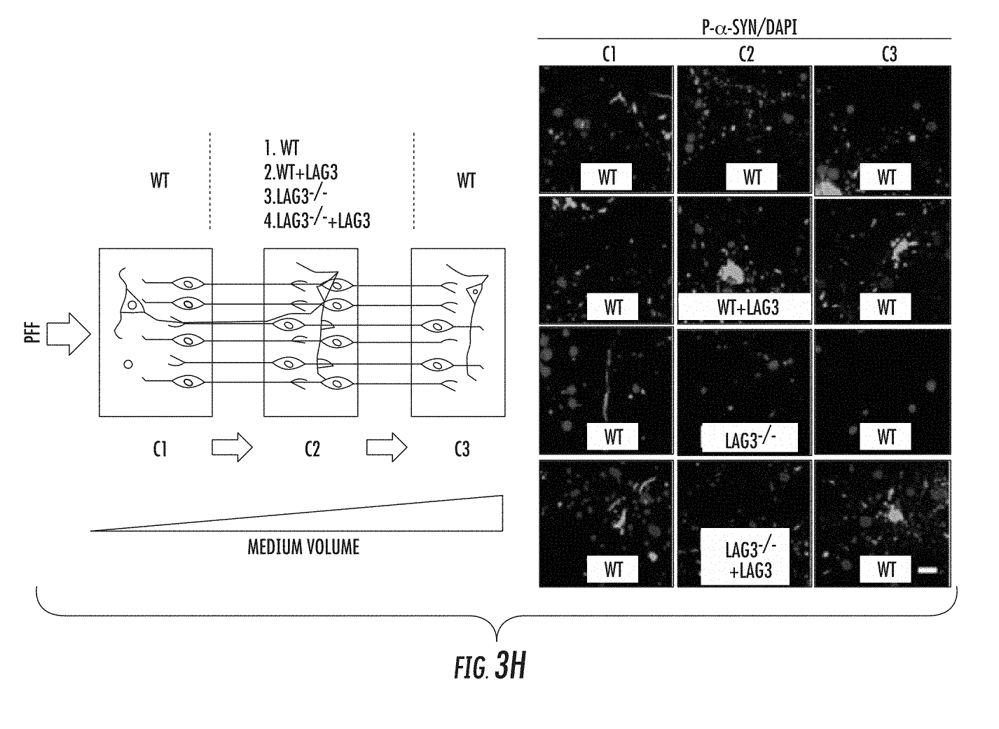

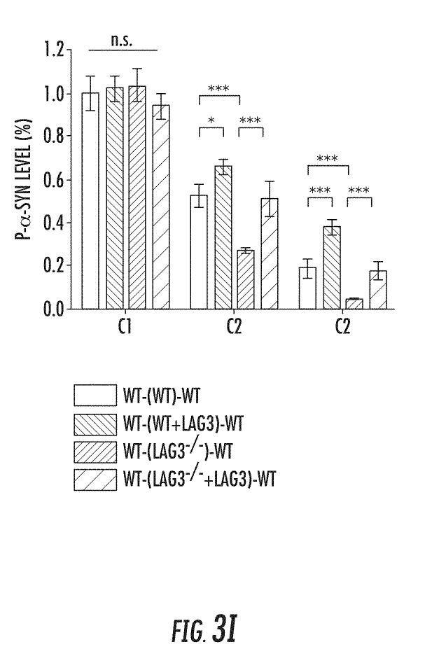

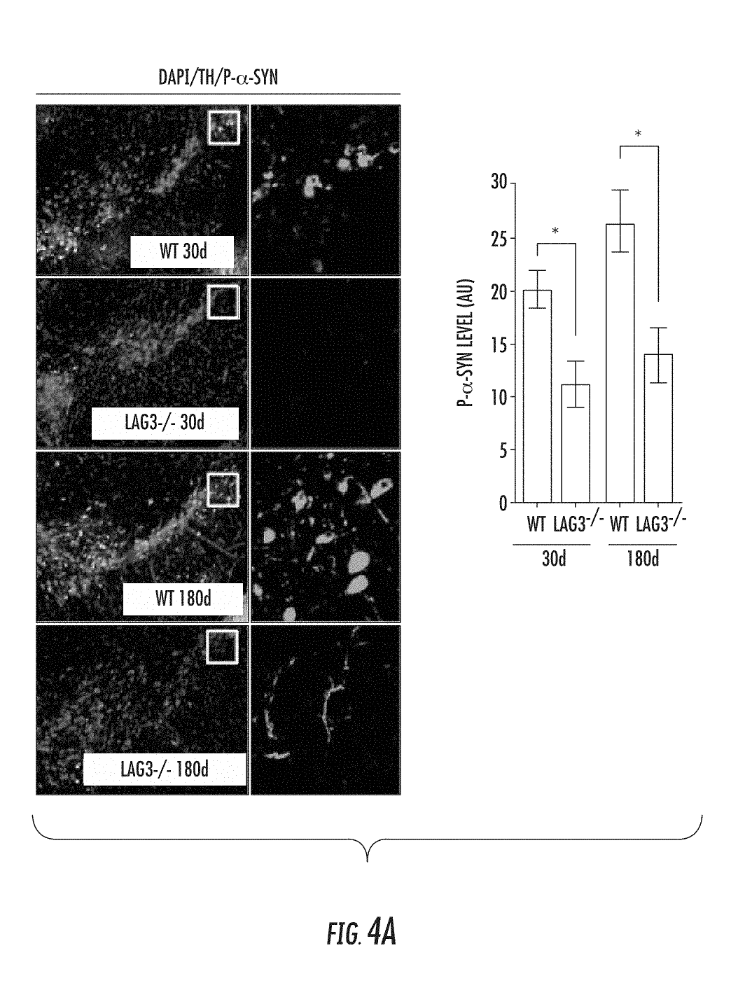

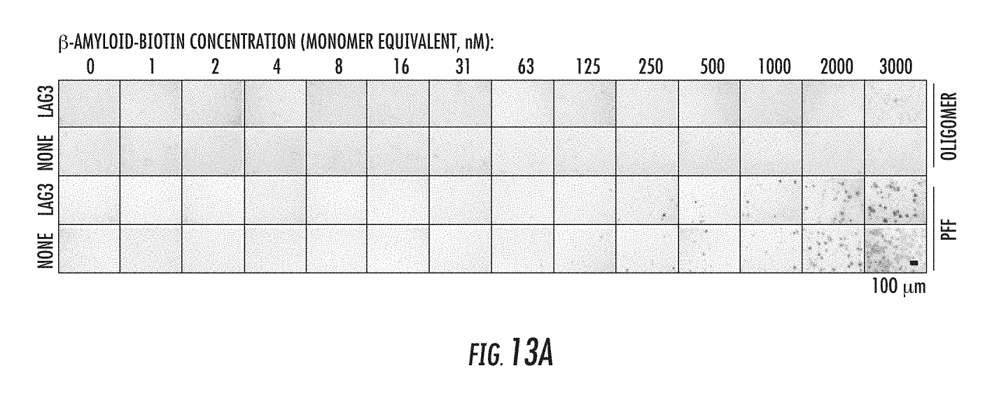

We then asked whether knocking out LAG3 prevents the pathology induced by α-syn PFF. Phosphorylation of α-syn at serine 129 (P-α-syn) and misfolded α-syn are associated with pathology in α-synucleinopathies. Their levels increase following administration of α-syn PFF to neuronal cultures. Accordingly, we administered α-syn PFF to wild type and LAG3 knockout cortical cultures at seven days in vitro (DIV). Ten days later the levels of P-α-syn are markedly increased in wild type cultures, while the levels of P-α-syn in LAG3 knockout cultures is barely detectable (FIG. 3A, 3B). Overexpression of LAG3 enhances the level of P-α-syn in wild type cultures and restores the levels in LAG3 knockout cultures (FIG. 3A, 3B). Overexpressing the D1 domain deletion mutant in LAG3 knockout neuron cultures fails to restore P-α-syn levels (FIG. 13A-13B). Overexpression of the D2, D3 and D4 domain deletion mutants in LAG3 knockout neuron cultures restore the α-syn pathology as monitored by P-α-syn (FIG. 13A-13B). An α-synuclein oligomeric/protofibrillar specific antibody (13) confirms that treatment of wild type cultures with α-syn PFF leads to aggregation of α-synuclein while the levels of oligomeric/protofibrillar α-syn are dramatically reduced in LAG3 knockout cultures compared to PBS treated cultures (FIG. 3A, 3B). Overexpression of LAG3 enhances the level of oligomeric/protofibrillar α-syn in wild type cultures and restores the level in LAG3 knockout cultures (FIG. 3A, 3B). Overexpression of the D1 domain deletion mutant in LGA3 knockout neuron cultures fails to restore oligomeric/protofibrillar α-syn levels (FIG. 13A-13B), while overexpression of the D2, D3 and D4 deletion mutants in LAG3 knockout neuron cultures restores the pathology (FIG. 13A-13B).

Immunoblots from lysates sequentially extracted in 1% TX-100 (TX-soluble) followed by 2% SDS (TX-insoluble) of α-syn and P-α-syn 12 days after α-syn PFF treatment of cortical neurons were examined. α-Syn PFF leads to an accumulation of α-syn and P-α-syn in the TX-insoluble fraction in wild type cultures, while there is significantly less accumulation in LAG3 knockout cultures (FIGS. 3C and 3D). α-syn PFF also causes a reduction in SNAP25 and synapsin II levels compared to PBS 12 days post-treatment as previously described (6). Deletion of LAG3 prevents the α-syn PFF-induced synaptic protein loss (FIGS. 3C and 3E). Overexpression of LAG3 in wild type cultures causes increased accumulation of α-syn and P-α-syn in the TX-insoluble fraction in wild type cultures and a further reduction in SNAP25 and synapsin II levels, whereas it prevents the sparing in LAG3 knockout cultures (FIGS. 3C and 3E).

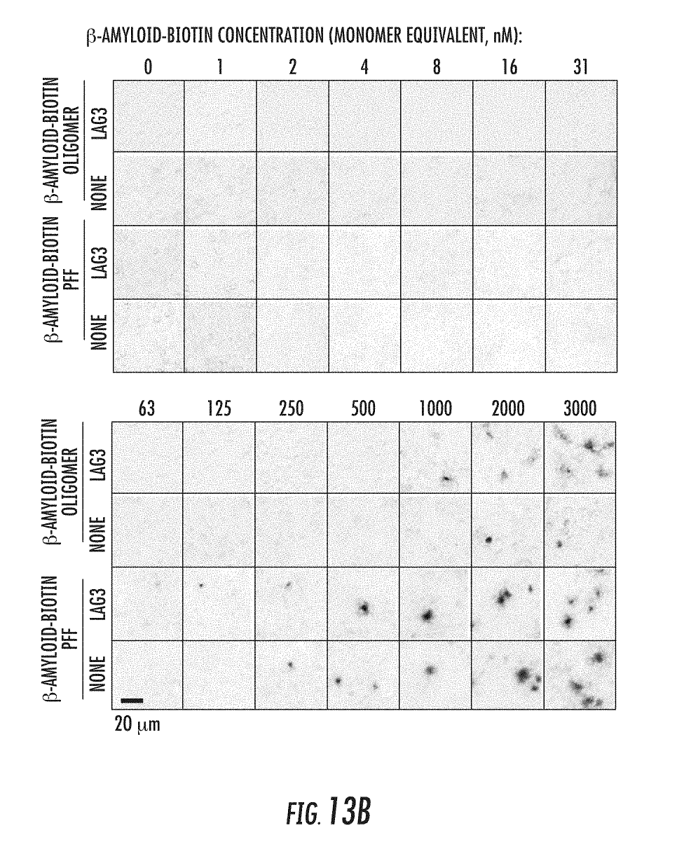

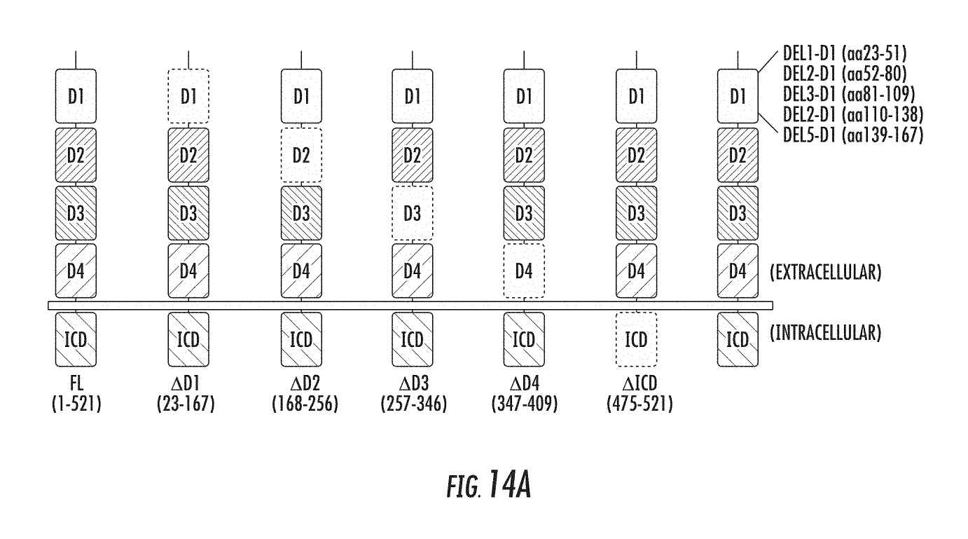

To examine the transmission of α-syn PFF and to establish the role of LAG3 in the intemeuron transmission of α-syn, we used a microfluidic neuronal culture device with three chambers connected in tandem by a series of microgrooves separating the chambers. The medium volume in chamber 1 (C1) is 50-μL lower than the one in chamber 2 (C2), and 100-μL lower than the one in chamber 3 (C3) to prevent diffusion of α-syn PFF to adjacent chambers. Cortical neurons were cultured in each chamber. To ensure that α-syn PFF cannot diffuse between chambers, primary wild type cortical neurons in C1 were treated with α-syn-biotin PFF. 14 days post-treatment, the neurons were fixed in 4% paraformaldehyde (PFA) and stained with streptavidin-568 fluorescence dye. Only neurons in C1 exhibit immunofluorescence, indicating that α-syn-biotin PFF cannot transmit from chamber to chamber through diffusion (FIG. 14A).

α-Syn transmission from C1 to C3 requires neurons in C2, since α-syn PFF administered to C1 fails to induce P-α-syn accumulation in C3 when C2 was left empty (FIG. 14B). Using this system, the transmission of α-syn PFF was monitored via P-α-syn levels in wild type and LAG3 knockout cultures (FIGS. 3F and 3G, FIG. 14C). The microfluidic neuron culture device was then set up to contain wild type cultures in C1 and C3, whereas C2 was varied and either contained wild type or LAG3 knockout cultures. In another set of chambers LAG3 was overexpressed in the C2 chamber containing either wild type or LAG3 knockout cultures. Administration of α-syn PFF to C1 leads to increased P-α-syn levels (FIGS. 3F and 3G, FIG. 14C). To assess the propagation of α-syn PFF along dendrites and axons as well as transmission of misfolded α-syn, the levels of P-α-syn was monitored in C2 and C3. When C2 contains wild type cultures, P-α-syn is observed in both C2 and C3 and LAG3 overexpression in C2 neurons enhances the levels of P-α-syn in both chambers (FIGS. 3F and 3G, FIG. 14C). In contrast, when C2 contains LAG3 knockout cultures, P-α-syn levels are significantly reduced in C2 and are absent in C3 (FIGS. 3F and 3G, FIG. 14C). LAG3 overexpression restores the propagation of α-syn PFF as assessed by similar levels of P-α-syn compared to wild type cultures (FIGS. 3F and 3G, FIG. 14C). These results taken together indicate that LAG3 is required for the propagation and transmission of pathologic α-syn.

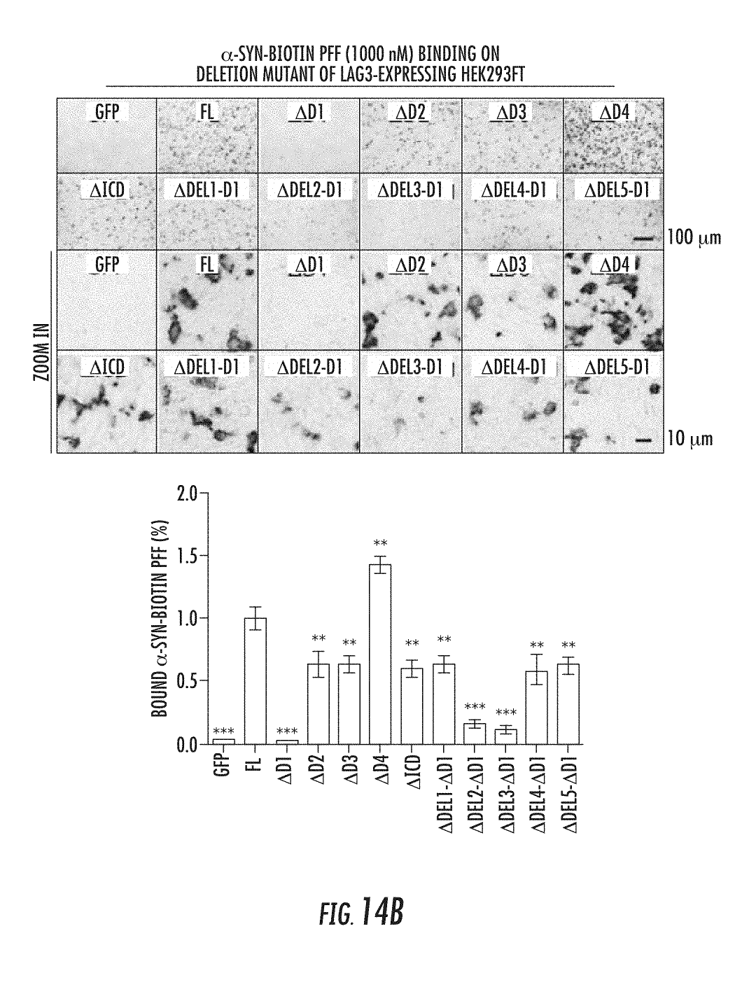

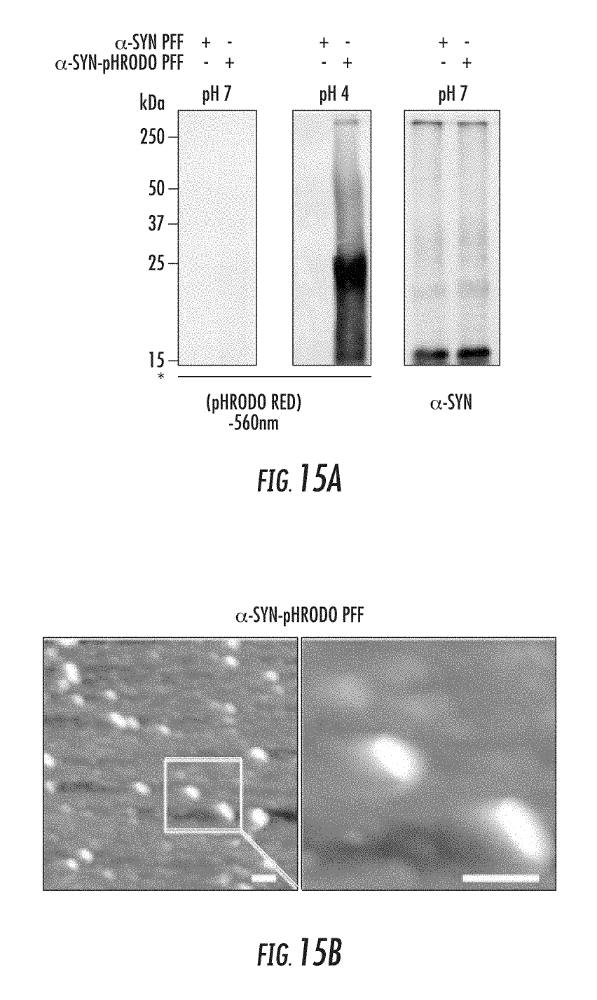

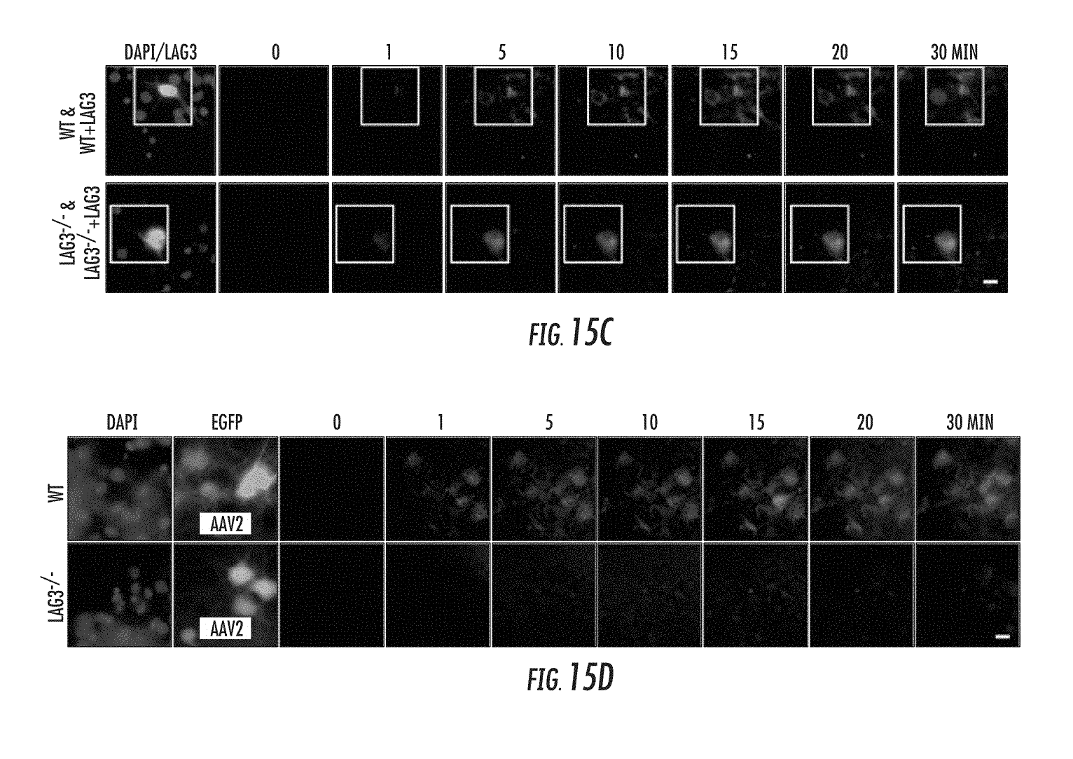

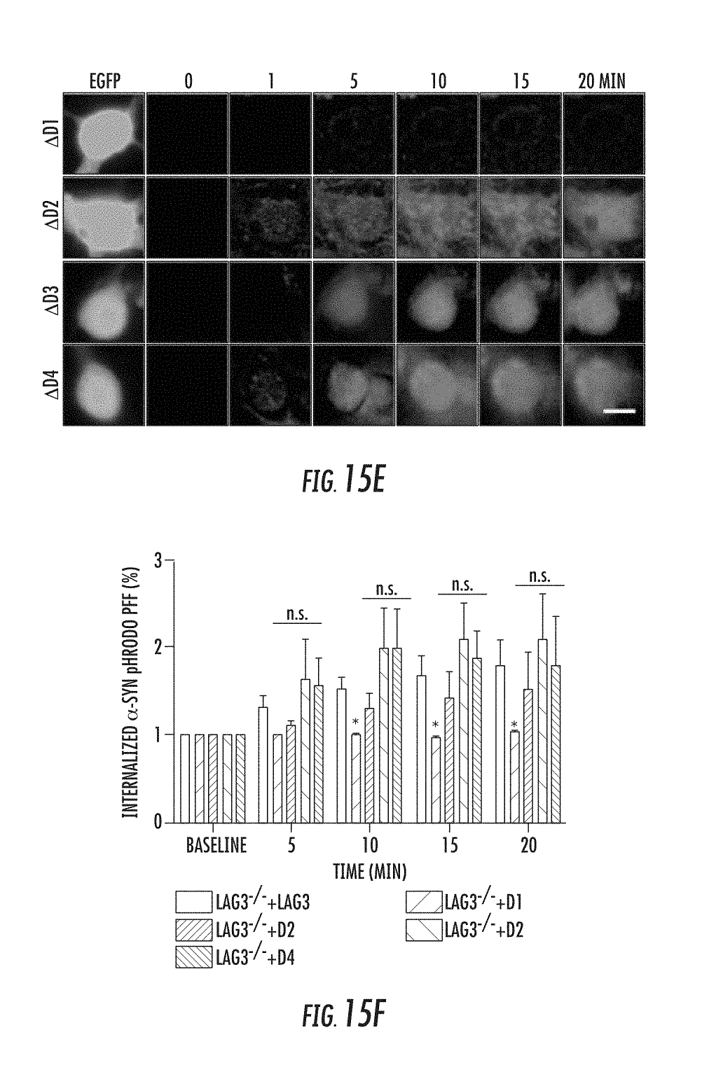

Treatment of wild type cortical cultures with α-syn PFF cause neuronal cell death as previously described (FIG. 15). α-Syn PFF treatment leads to substantial cell death compared to PBS treated cultures as assessed by propidium iodide staining (FIGS. 15A and 15B). LAG3 knockout cultures exhibit significantly less cell death and overexpression of LAG3 restores the toxicity to α-syn PFF (FIG. 15). Neuronal nuclei (NeuN) antibody staining was also performed to assess neuronal degeneration. α-Syn PFF treatment causes a significant loss of NeuN immunoreactivity and overexpression of LAG3 enhances the loss (FIGS. 15C and 15D). NeuN immunoreactivity is preserved in LAG3 knockout cultures after α-syn PFF treatment, whereas overexpression of LAG3 in knockout cultures leads to a loss of NeuN immunoreactivity (FIGS. 15C and 15D). NeuN immunostaining of deletion mutants (ΔD1-D4, ΔICD) overexpression in LAG3 knockout neurons indicates that deletion of the D1 domain fails to exhibit cell death, but deletion of D2, D3, D4 or the ICD domains still lead to cell death (fig. S11E).

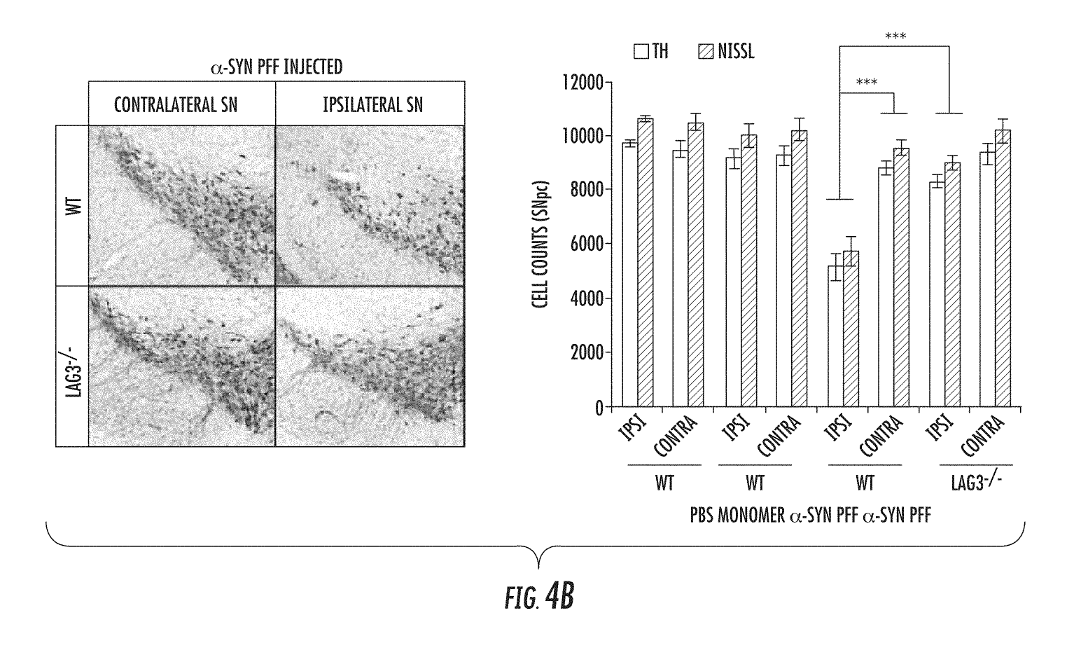

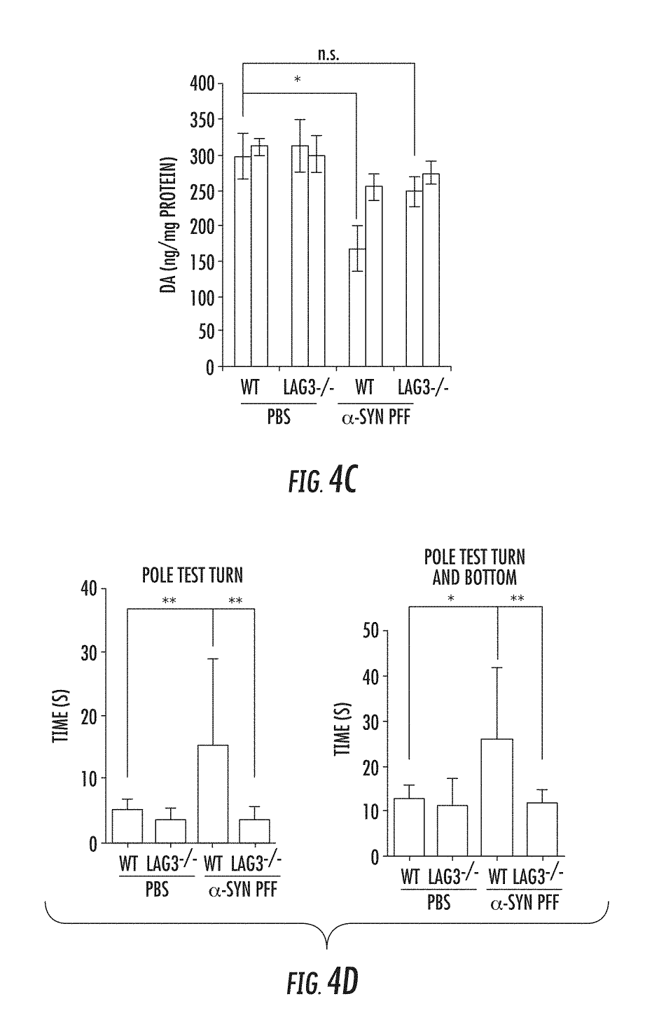

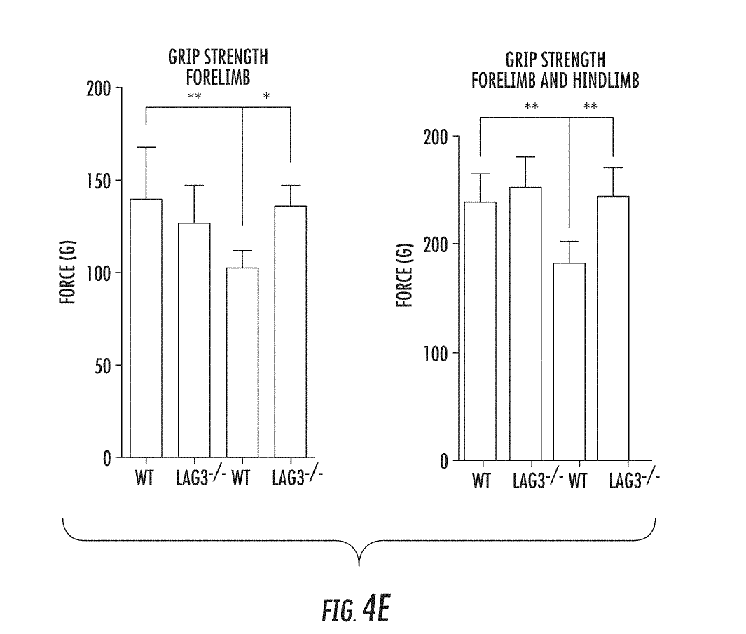

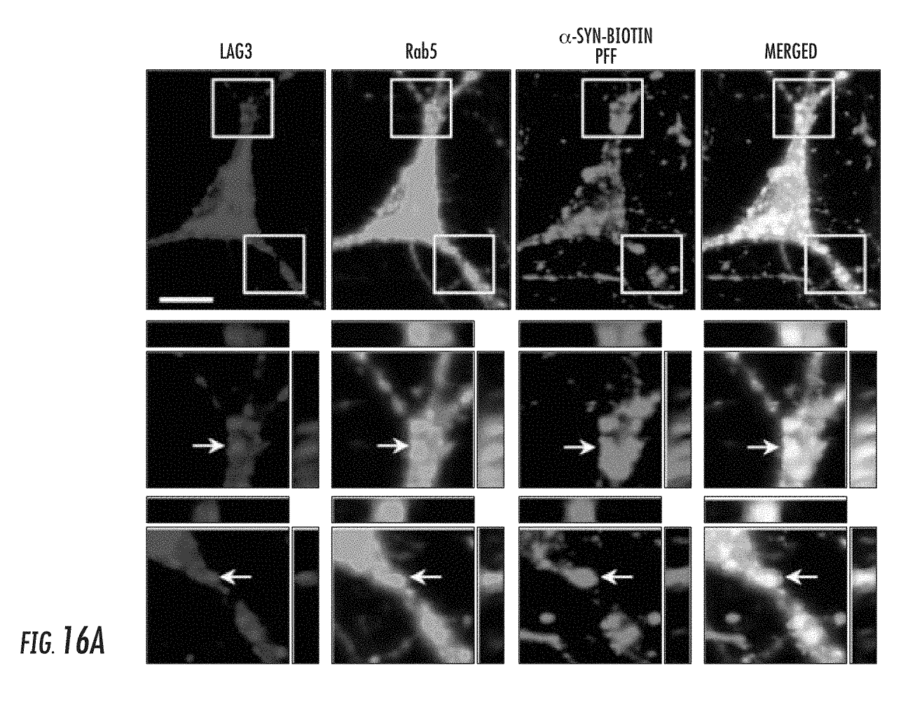

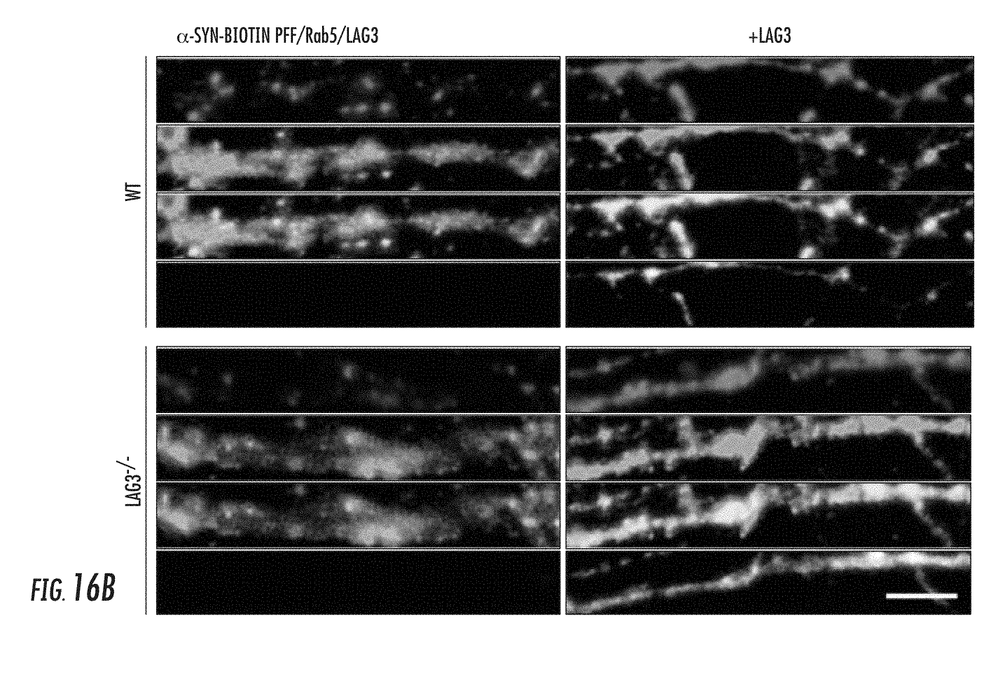

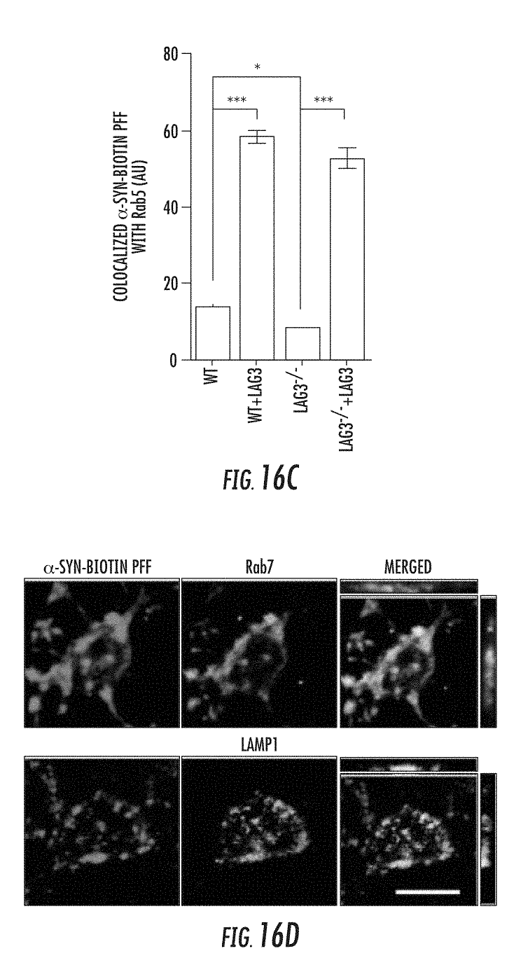

To determine whether LAG3 is necessary for α-syn PFF transmission and toxicity in vivo, α-syn PFF were stereotactically injected into the dorsal striatum of wild type and LAG3 knockout mice (FIG. 16A). Representative maps of LB/LN-like pathology of P-α-syn accumulation (red dots) and the stereotaxic injection site indicated by gray circles in the α-syn PFF-injected hemisphere are shown for mice sacrificed at 30 and 180 dpi (fig. S12A). P-α-syn immunoreactivity was monitored in tyrosine hydroxylase (TH) neurons thirty and 180 days after α-syn PFF injection. We observe substantial P-α-syn staining in wild type TH positive neurons at 30 and 180 days (FIG. 4A). In LAG3 knockout TH positive neurons, P-α-syn staining is reduced by greater than 50% at both time points. Accompanying the α-syn pathology, stereologic counting of TH and Nissl positive neurons reveals significant loss of dopamine neurons in wild type mice at 180 days post-injection (FIG. 4B). There is a dramatic preservation of dopamine neurons in α-syn PFF injected LAG3 knockout mice (FIG. 4B). HPLC analysis demonstrates a significant reduction in dopamine and its metabolites DOPAC and HVA in wild type mice and a sparing of the reduction in LAG knockout mice (FIG. 4C and FIG. 16B, 16C, 16D). Immunoblot analysis demonstrates a significant reduction in TH and DAT in wild type mice and a sparing of the reduction in LAG knockout mice (FIG. 16E). At 180 days post-α-syn PFF injection the wild type mice exhibit robust clasping behavior when suspended by their tail, whereas LAG KO mice demonstrate a response similar to PBS injected mice (FIG. 16F). Wild type mice show significant impairment in the pole test, which is thought to be a sensitive behavioral indicator of dopaminergic function, with increase time to turn and time to reach the base, whereas LAG knockout mice show no significant impairments (FIG. 4D). Grip strength analysis indicates that wild type mice have reduced forelimb and forelimb and hindlimb strength after α-syn PFF injection, while the LAG knockout mice show no significant loss in grip strength (FIG. 4E). Therefore, LAG3 is crucial for α-syn PFF induced neurodegeneration and development of PD related motor defects. The present invention has identified LAG3 as the transmission receptor for α-syn PFF that mediates the deleterious effects of misfolded α-syn that can be used to develop therapeutic agents. We isolated LAG3 via an unbiased screen for α-syn PFF binding sites. Although our data indicates that LAG3 is not the sole α-syn PFF binding site, it is essential for α-syn PFF endocytosis and transmission. Moreover, mice lacking LAG3 are resistant to the toxic effects of α-syn PFF.

Recently, the Toll-like receptor 2 (TLR2) on microglia was shown to be involved in the activation of microglia due to exposure to oligomeric α-syn from conditioned neuronal media. On the other hand, LAG3 appears to mediate the transmission of misfolded α-syn from neuron to neuron. According to the Allen Brain Atlas, it is localized to neurons throughout the central nervous system including dopamine neurons. The function of LAG3 in the CNS is not known and whether misfolded α-syn activates downstream signaling following engagement of LAG3 requires further study.

Recent studies have revealed that lymphocytes can extract surface molecules from antigen presenting cells through a process called trogocytosis. Lag3 is enriched in lymphocytes and binds to major histocompatibility complex (MHC) class II from neighboring cells where it may participate in trogocytosis. Trogocytosis has been proposed a mechanism for intercellular communication either through endocytic vesicles or through a membrane bridge. We propose a novel mechanism cell-to-cell transmission of misfolded α-syn that involves the endocytosis of exogenous α-syn PFF by the engagement of LAG3 on neurons similar to Lag3 facilitated trogocytosis by binding to MHC class II molecules.

In summary, the interaction between LAG3 and α-syn PFF provides a new target for the development of therapeutics designed to slow the progress of PD and related α-synucleinopathies.

Embodiments of the disclosure concern methods and/or compositions for treating and/or preventing a neurological disorder in which modulation of the α-syn PFF transmission pathway is directly or indirectly related. In certain embodiments, individuals with a neurological disorder such as Parkinson's disease (PD) are treated with an agent that acts as a modulator of the pathway, and in specific embodiments an individual with PD is provided an agent that inhibits α-syn PFF from binding to its receptor and/or neural transmission of α-syn PFF.

In certain embodiments, the level to which an agent inhibits α-syn PFF from binding to its receptor and/or neural transmission of α-syn PFF may be any level so long as it provides amelioration of at least one symptom of the neurological disorder, for example PD. The level of inhibition may increase by at least 2, 3, 4, 5, 10, 25, 50, 100, 1000, or more fold compared to the level in a standard (where the agent is not applied), in at least some cases.

An individual known to have PD, suspected of having PD, or at risk for having PD may be provided an effective amount of an agent of the present invention. Those at risk for PD may be those individuals having one or more genetic factors, may be of advancing age, and/or may have a family history, for example.

In particular embodiments of the disclosure, an individual is given a second or third agent for PD therapy in addition to the one or more agents that inhibit α-syn PFF from binding to its receptor and/or neural transmission of α-syn PFF. Such additional therapy may include L-DOPA or dopamine receptor agonists and/or deep brain stimulation, for example. When combination therapy is employed with one or more agents that inhibit α-syn PFF from binding to its receptor and/or neural transmission of α-syn PFF the additional therapy may be given prior to, at the same time as, and/or subsequent to the agent that inhibits α-syn PFF from binding to its receptor and/or neural transmission of α-syn PFF.

Certain methods of the disclosure provide for methods of diagnosing PD prior to the therapeutic methods of the disclosure, and such diagnosis may occur by any methods or means, including at least genetic marker assay, single-photon emission computed tomography, olfactory system testing, autonomic system testing, or a combination thereof.

### Pharmaceutical Preparations

Pharmaceutical compositions of the present invention comprise an effective amount of one or more agents that inhibit α-syn PFF from binding to its receptor and/or neural transmission of α-syn PFF dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one agent that inhibits α-syn PFF from binding to its receptor and/or neural transmission of α-syn PFF or additional active ingredient will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The agent that inhibits α-syn PFF from binding to its receptor and/or neural transmission of α-syn PFF may comprise different types of carriers depending on whether it is to be administered in solid, liquid or aerosol form, and whether it need to be sterile for such routes of administration as injection. The present compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The agent that inhibits α-syn PFF from binding to its receptor and/or neural transmission of α-syn PFF may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like. Further in accordance with the present disclosure, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include an agent that inhibits α-syn PFF from binding to its receptor and/or neural transmission of α-syn PFF one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the agent that inhibits α-syn PFF from binding to its receptor and/or neural transmission of α-syn PFF may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### A. Alimentary Compositions and Formulations

In one embodiment of the present disclosure, the agents that inhibit α-syn PFF from binding to its receptor and/or neural transmission of α-syn PFF are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### B. Parenteral Compositions and Formulations

In further embodiments, agents that inhibit α-syn PFF from binding to its receptor and/or neural transmission of α-syn PFF may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### C. Miscellaneous Pharmaceutical Compositions and Formulations

In other preferred embodiments of the invention, the active compound or agent that inhibits α-syn PFF from binding to its receptor and/or neural transmission of α-syn PFF may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### Kits of the Disclosure

Any of the compositions described herein may be comprised in a kit. In a non-limiting example, an agent that inhibits α-syn PFF from binding to its receptor and/or neural transmission of α-syn PFF may be comprised in a kit.

The kits may comprise a suitably aliquoted agent that inhibits α-syn PFF from binding to its receptor and/or neural transmission of α-syn PFF and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the agent that inhibits α-syn PFF from binding to its receptor and/or neural transmission of α-syn PFF and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The agent that inhibits α-syn PFF from binding to its receptor and/or neural transmission of α-syn PFF (s) may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

### Examples/Methods

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

α-Syn purification and PFF preparation. Recombinant α-syn was purified as published. Assembly reactions were agitated in a transparent glass vial with a magnetic stirrer (350 rpm at 37° C.). After 7 days incubation and then sonication, α-syn monomer/PFF was separated by HPLC and kept in −80° C. To characterize α-syn PFF receptors, recombinant α-syn monomer was purified and labeled with sulfo-NHS-LC-Biotin (Thermo Scientific, EZ-link Sulfo-NHS-LC-Biotin, 21435). The mole ratio of biotin to α-syn was 2-3. After conjugation, α-syn-biotin monomer/PFF is prepared as mentioned above.
Expression cloning and SH-SY5Y cell surface binding assays. We performed a directed experiment to identify α-syn PFF receptor(s): a library consisting of 352 individual preparations of cDNAs encoding transmembrane proteins (TMGW10001, GFC-transfection array panel, Origene) was transfected into SH-SY5Y cells. Two days after transfection, the cells were incubated with α-syn-biotin PFF (1 μM total α-syn-biotin monomer concentration) in DMEM media with 10% FBS at 22° C. for 2 h. Next, unbound α-syn-biotin PFF was removed by extensive washing with DMEM with 10% FBS. The cells were fixed with 4% paraformaldehyde in PBS, washed three times with PBS, blocked for 30 min with 10% goat serum and 0.1% Triton X-100 in PBS, and incubated for 16 h with alkaline-phosphatase-conjugated streptavidin in PBS supplemented with 5% goat serum and 0.05% Triton X-100. Finally, bound alkaline-phosphatase was visualized by 5-bromo-4-chloro-3-indolyl phosphate/nitro blue tetrazolium reaction. Quantification of bound α-syn-biotin PFF to LAG3-transfected SH-SY5Y cells was performed with ImageJ. We also tested similarly several candidate receptors; the cDNA plasmids were obtained from Addgene.
Primary neuronal cultures, α-syn PFF transduction and neuron binding assays. Primary cortical neurons were prepared from E15.5 and cultured in Neurobasal media supplemented with B-27, 0.5 mM L-glutamine, penicillin and streptomycin (all from Invitrogen) on tissue culture plates coated with poly-L-lysine. The neurons were maintained by changing medium every 3-4 days. α-syn PFF transduction was performed at 7 DIV and α-syn PFF was kept for 10-21 days for biochemical experiment or toxicity assay. Each experiment was performed in duplicate and repeated 3-6 times. Transduced neurons were harvested for indirect immunofluorescence and sequential extraction. To determine bound α-syn-biotin PFF in wild type and LAG3 knockout culture, α-syn-biotin PFF with different indicated concentrations were used. Quantification of bound α-syn-biotin PFF to wild type and LAG3 knockout neurons were performed with ImageJ.
ELISA analysis. The binding affinity between α-syn-biotin PFF and LAG3 were analyzed using a sandwich ELISA kit (Sigma) according to manufacturer instructions. The lyophilized human LAG3 protein was added into a human LAG3 antibody-coated ELISA plate and left overnight at 4° C. with gentle shaking. After the extensive wash, different concentrations of α-syn-biotin PFF (0.1 nM to 100 nM) were added to each well and were incubated for 2 hours at 22° C. with gentle shaking. HRP-streptavidin solution was incubated for 45 min at 22° C. with gentle shaking and follows with the extensive wash. Finally, ELISA colorimetric TMB Reagent was incubated for 10 min at 22° C. in the dark with gentle shaking.
Plasmids. Human and mouse LAG3 cDNA clones were kindly obtained from Dr. Charles Drake at the Johns Hopkins University, School of Medicine. APLP1 cDNA clone was obtained from Dr. Yasushi Shimoda at Nagaoka University of Technology and Dr. Gopal Thinakaran at The University of Chicago. Neurexin cDNA clones were obtained from Dr. Thomas C. Sildhof at Stanford University and Dr. Peter Scheiffele at Basel University. The rest cDNA plasmids were obtained from Addgene. Deletion mutants. LAG3 deletion mutants were constructed by PCR using herculase polymerase (Agilent Technologies) and primers flanking the sequences to be deleted. The DNA was separated on a 1% agarose gel and the appropriate band was isolated using a gel extraction kit (Qiagen). 100 ng of DNA was phosphorylated at the 5′ end using T4 polynucleotide kinase (Invitrogen) for 30 mins at 37° C. and ligated overnight at room temperature using T4 DNA ligase (Invitrogen). Reactions were purified with a PCR purification kit (Qiagen) and transformed into competent Stbl3 cells (Invitrogen).
Live images and confocal microscopy. α-Syn PFF was labeled with pHrodo red (Invitrogen). pHrodo red is weakly fluorescent at neutral pH but increasingly fluorescent as the pH drops. α-syn-pHrodo PFF was directly added to LAG3 wild type (WT) and knockout (KO) neuron groups. For the WT+LAG3 and KO+LAG3 groups, neurons were transfected with LAG3 expression vector 2 days prior to the addition of α-syn-pHrodo PFF. Live images were observed every minute for 20 minutes using confocal microscopy. To confirm the endocytosis of α-syn PFF mediated by LAG3, neurons were fixed 2 hour after α-syn-biotin PFF treatment using 4% paraformaldehyde in PBS.
Microsome enrichment. α-syn-biotin PFF was administrated into the neuron (12 DIV) cultures and incubated for 1.5 h. To clear up the bound α-syn-biotin PFF, trypsin was added for 30 seconds and follows with 3 times medium wash. The neurons were harvested with PBS and prepared with the lysis buffer (250 mM sucrose, 50 mM Tris-C1 (pH 7.4), 5 mM MgCl₂, 1 mM EDTA, 1 mM EGTA) with inhibitor cocktail. The suspended cell lysates were pipetted for 6 times and syringed for 20 times. The microsomes were harvest in the third pellet following by three steps of centrifuges 1^{st} (1000 g, 10 min), 2^{nd} (16,000 g, 20 min) 3^{rd} (100,000 g, 1 h) for immunoblot directly.
Biochemical analysis. Dissected brain regions of interest or culture samples were prepared with RIPA buffer (50 mM Tris [pH 8.0], 150 mM NaCl, 1% Triton-100, and 2% SDS) containing protease and phosphatase inhibitor (Roche). Samples were sonicated and centrifuged with 20,000 g for 20 min. Protein concentrations were determined using the BCA assay (Pierce) and samples (10 g total proteins) were separated on SDS-polyacrylamide gels (13.5%) and transferred onto nitrocellulose membranes. Blots were blocked in 5% non-fat milk or 7.5% BSA in TBST and probed using various primary antibodies. Target antigens were detected using ImageQuant LAS 4000mini scanner or film following incubation with the appropriate infrared secondary antibodies.
In vivo co-immunoprecipitation. Transgenic mice overexpressing human A53T α-synuclein, as previously characterized (9), and wild type littermate controls were sacrificed at 4 months and 8 months of age. The brainstem was removed and lysates prepared with brain lysis buffer containing 50 mM Tris [pH 8.0], 150 mM NaCl, 1% NP-40, and protease inhibitors (Roche). Samples were frozen and thawed three times, followed by centrifugation at 14,000 rpm for 20 min. Protein concentration of the supernatants were determined using the BCA assay (Pierce). Aliquots of the samples containing 500 μg of protein were pre-cleared with 10 μL of Dynabeads® Protein G (Life Technologies) for one hour. Simultaneously, 50 μL of Dynabeads® were incubated for one hour with 4 μL of either rabbit α-synuclein antibody (Cell Signaling) or rabbit Igg (Santa Cruz). Pre-cleared samples were incubated with Dynabead®-antibody/Igg overnight at 4° C. The immunocomplexes were washed five times with IP buffer and then denatured by adding 2× Laemlli Buffer plus β-mercaptoethanol, followed by boiling for five minutes.
Microfluidic chambers. Triple compartments microfluidic devices were obtained from Xona Microfluidic (TCND 1000). Glass coverslips were prepared and coated as described before being affixed to microfluidic devices (6). Approximately 100,000 neurons were plated per chamber. At 4 DIV, WT+LAG3 and KO+LAG3 groups were transduced with lenti-virus LAG3. At 7 DIV, 0.5-μg α-syn PFF was added into chamber 1. To control for direction of flow, a 75-μL difference in media volume was maintained between Chamber 1 and Chamber 2, Chamber 2 and Chamber 3 according to the manufacturers' instructions. Neurons were fixed 14 days after α-syn PFF treatment using 4% paraformaldehyde in PBS. The devices were then ready to be used for immunofluorescence staining.
Mouse strains. C57BL6 and CD1 mice were obtained from the Jackson Laboratories (Bar Harbor, Me.). LAG3 knockout mice were kindly obtained from Dr. Drake in Johns Hopkins University and were maintained on a C57BL6 background. All housing, breeding, and procedures were performed according to the NIH Guide for the Care and Use of Experimental Animals and approved by the Johns Hopkins University Animal Care and Use Committee.
Injection material and stereotaxic injections. Purification of recombinant of α-syn proteins and in vitro fibril assembly was performed as published (8). Assembly reactions were agitated in a transparent glass vial with a magnetic stirrer (350 rpm at 37° C.) and PFF harvested after 7 days. Preparations were diluted in sterile PBS and sonicated briefly with a hand held probe before intracerebral injection. Mice between 2 and 3 months of age were anesthetized with pentobarbital (diluted 1:4 in sterile saline, 250 μL for 25 mg mice) and stereotactically injected in one hemisphere with recombinant α-syn PFF (5 μg). Control animals received sterile PBS. A single needle insertion (coordinates: +0.2 mm relative to Bregma, +2.0 mm from midline) into the right forebrain was used to target the inoculum to the dorsal neostriatum (+2.8 mm beneath the dura). Injections were performed using a 2 μL syringe (Hamilton, Nev.) at a rate of 0.1 μL per min (2.5 μL total per site) with the needle in place for ≥5 min at each target. Animals were monitored regularly following recovery from surgery, and sacrificed at various pre-determined time points (30 or 180 dpi) by overdose with pentobarbital. For histological studies the brains were removed after transcardial perfusion with PBS and 4% PFA, and underwent overnight fixation in 4% PFA, cryoprotected in 30% sucrose. For biochemical studies, tissues were immediately frozen after removal and stored at −80° C. until used.
Behavioral Analysis. To evaluate the effects of transmissible α-syn pathology on motor skills, mice were tested with three behavioral tests 1-week prior to sacrifice. The order of tests was randomized and an experimenter blinded to treatment group conducted all tests. All tests were conducted between 10:00-16:00 in the lights-on cycle. Mice were habituated to the testing room 1 day before tests, and the apparatus were cleaned with 70% ethanol in between animals to minimize odor cues.

TABLE S1
Antibodies used in this study.

| Antibodies | Source/Cat. No./Ref. | Host | Dilution |
|---|---|---|---|
| α-Syn | BD Biosciences (610787) | Mouse | 1:2000 (WB) |
| | Cell Signaling (2642) | Rabbit | 4 μL/Sample (IP) |
| P-α-syn Ser129 | Abcam (ab168381) | Rabbit | 1:1000 (IF/IHC) |
| | Sigma (SAB4300139) | Rabbit | 1:1000 (WB) |
| | Convance | Mouse | 1:1000 (IF/IHC) |
| Tyrosine | Sigma (T2928) | Mouse | 1:1000 |
| Hydroxylase | | | (WB/IHC/IF) |
| (TH) | | | |
| Dopamine | Sigma (D6944) | Rabbit | 1:1000 (WB) |
| transporter | | | |
| (DAT) | | | |
| Synapsin II | Abcam (ab13258) | Rabbit | 1:1000 (WB) |
| SNAP25 | Cell Signaling (D7B4) | Rabbit | 1:1000 (WB) |
| LAG3 | eBioscience (C9B7W) (10) | Rat | 1:100 (exp) |
| | 410C9 (19) | Mouse | 1:1000 (IP/WBIF) |
| | Anti-D1 (29) | Rabbit | 1:1000 (WB) |
| Rab5 | Abcam (ab18211) | Rabbit | 1:1000 (IF) |
| Rab7 | Cell Signaling (2094S) | Rabbit | 1:1000 (WB/IF) |
| LAMP1 | Abcam (ab24170) | Rabbit | 1:1000 (IF) |
| Clathrin | Abcam (Ab59710) | Rabbit | 1:1000 (IF) |
| MAP2 | Sigma (M9942) | Mouse | 1:1000 (WB/IF) |
| IBA-1 | Abcam (ab5076) | Goat | 1:500 (WB) |
| GFAP | Abcam (ab7260) | Rabbit | 1:1000 (WB) |
| NeuN | Millipore (MAB377) | Mouse | 1:1000 (IF) |

Table S2. cDNA Library Data not Shown.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of inhibiting neurodegeneration in a subject comprising administering to the subject an agent that prevents α-syn PFF from binding to its receptor.

2. The method of claim 1 wherein the agent is a capture molecule.

3. The method of claim 1 wherein the receptor is lymphocyte-activation gene 3 (LAG3).

4. The method of claim 1 wherein the receptor is selected from the group consisting of Neurexin1β, Neurexin2β, Neurexin3β, or a combination thereof.

5. The method of claim 1 wherein the receptor is amyloid precursor-like protein 1 (APLP1).

6. The method of claim 1 wherein the receptor is a human receptor.

7. The method of claim 2 wherein the capture molecule binds to LAG3.

8. The method of claim 2 wherein the capture molecule binds to the LAG3 D1 domain.

9. The method of claim 2 wherein the capture molecule binds to amino acids 81-109 of the D1 domain of LAG3.

10. The method of claim 9 wherein the capture molecule binds to amino acids 52-80 of the D1 domain of LAG3.

11. The method of claim 1 wherein the subject comprises α-syn PFF and endocytosis of α-syn PFF is inhibited in the subject.

12. The method of claim 1 wherein the agent is a vector that expresses antisense LAG3 mRNA in the subject.

13. The method of claim 1 wherein the phosphorylation of α-syn at serine 129 is inhibited in the subject.

14. The method of claim 1 wherein the misfolding of α-syn protein is inhibited in the subject.

15. The method of claim 1 wherein the subject has Parkinson's disease.

16. The method of claim 2 wherein the capture molecule is selected from the group comprising an antibody, an antibody fragment, an aptamer, and a monoclonal antibody.

17. A method of drug screening comprising the steps of:
providing one or more agent(s);
applying the one or more agents to LAG3, and
identifying those agents that prevent α-syn PFF from binding to LAG3.

18. The method of claim 17 wherein the agent is selected from the group consisting of a small chemical compound, antibody, nucleic acid, polypeptide, or combination thereof.

19. A method of drug screening comprising the steps of:
providing one or more agent(s);
applying the one or more agents to cells, and
identifying those agents that prevent α-syn PFF from binding to LAG3 or that inhibit the phosphorylation of α-syn PFF at serine 129.

20. The method of claim 1 wherein the subject has Parkinson's disease and the method treats or prevents Parkinson's disease.

21. The method of claim 1 wherein the subject has Diffuse Lewy Body Disease (DLB) and the method treats or prevents Diffuse Lewy Body Disease.

22. The method of claim 1, wherein the subject has dementia with Lewy Bodies and the method treats or prevents dementia with Lewy Bodies.

23. The method of claim 1 wherein the subject has multiple atrophy and the method treats or prevents multiple atrophy.
